(19) United States
(12) Patent Application Publication
(10) Pub. No.: US 2014/0288278 A1
(43) Pub. Date: Sep. 25, 2014

(54) CHROMATOGRAPHY PROCESS FOR RESOLVING HETEROGENEOUS ANTIBODY AGGREGATES

(71) Applicants: Joseph Nti-gyabaah, Somerset, NJ (US); Sandra Meissner, Secaucus, NJ (US); Rebecca Chmielowski, Scotch Plains, NJ (US); Janelle Konietzko, Lansdale, PA (US)

(72) Inventors: Joseph Nti-gyabaah, Somerset, NJ (US); Sandra Meissner, Secaucus, NJ (US); Rebecca Chmielowski, Scotch Plains, NJ (US); Janelle Konietzko, Lansdale, PA (US)

(21) Appl. No.: 14/355,014

(22) PCT Filed: Oct. 25, 2012
(86) PCT No.: PCT/US12/61833

§ 371 Date: Apr. 29, 2014

Related U.S. Application Data
(60) Provisional application No. 61/553,568, filed on Oct. 31, 2011.

(51) Int. Cl. C07K 16/24; C07K 1/16

(52) U.S. Cl. CPC C07K 16/241; C07K 1/165
USPC 530/388.24; 530/388.1

(57) ABSTRACT

Disclosed are methods and processes utilizing multi-modal chromatography as a polishing step to separate heterogeneously charged (basic and acidic) aggregates and other impurities from partially a partially purified bulk product monoclonal antibody product. The resulting chromatographic process provides a scaleable production process that is cost effective and increases the productivity of the purification process over a platform utilizing a combination of anion and cation exchange chromatography to resolve heterogeneous aggregates.

### BACKGROUND OF THE INVENTION

The need for high throughput purification processes for monoclonal antibodies (mAbs) has been heightened with the development of improved cell culture methods resulting in an increased mass of product to be purified. Furthermore, the desire to produce a high purity mAb is also heightened because aggregates can change protein activity and create potentially undesirable immunological responses in patients. Monoclonal antibody aggregates are typically resolved from a partially purified mAb drug product composition via cation exchange chromatography methods due to their basic charge.

Protein aggregation is a common phenomenon that can be encountered during various stages of a commercial antibody manufacturing processes. For example, aggregates can form during the fermentation, purification, final formulation operations, or as a result of the storage of the drug substance or final drug product. Usually aggregation results from intermolecular associations of partially denatured protein chains, however it may also result from chemical degradation and subsequent exposure of hydrophobic surfaces, or from disulfide bond scrambling. Mono-dispersity of a therapeutic monoclonal antibody (mAb) is important in terms of both efficacy and safety. Molecular heterogeneity in size (e.g. aggregation) can comprise the biological activity of a monoclonal antibody resulting in a partial or total loss of its therapeutic properties. In addition, the presence of aggregates in a biopharmaceutical product provides the potential for an immunogenic reaction which can diminish the efficacy of the product or, worse, cause a severe hypersensitivity response which can endanger the health of patients who are intended to benefit from antibody therapy.

Downstream processing has been estimated to account for 50 to 80% of the total manufacturing costs of therapeutic antibodies. Numerous strategies have been developed to minimize antibody aggregation during the upstream and downstream unit operations required for antibody manufacturing including optimization of media components, culture fed conditions and operating parameters, genetic engineering of host cell/expression systems, protein engineering, formulation buffer screening and separation during down-stream processing. However, it is widely recognized that it may not be possible, or commercially feasible, to optimize a particular manufacturing process to the point where protein aggregation is completely suppressed or prevented during manufacturing. Accordingly, separation of aggregates from drug substance using a downstream manufacturing step (unit operation) that is optimized for a particular protein of interest (POI) is a popular strategy which affords an opportunity to remove aggregates from the drug substance once they have been generated.

Therefore, in order to effectively manage the issue of size heterogeneity during the manufacturing of biopharmaceuticals, including therapeutic antibodies, there is an unmet need for alternative chromatography processes which are capable of separating monomeric antibody from aggregates and complexes which may form as a result of process-driven modifications or manufacturing conditions.

### SUMMARY OF THE INVENTION

The present invention relates to the identification and optimization of polishing steps suitable for use in the purification of monoclonal antibodies characterized by heterogeneous aggregates. A combination of high throughput screening (HTS) and design of experiment (DoE) strategies were employed to rapidly evaluate the use of different chromatographic modalities as a final polishing step for the purification a CHO-produced biosimilar monoclonal antibody drug product which is characterized by conditioned mixtures comprising heterogeneous aggregates. As disclosed herein, the use of a mixed mode (e.g., Capto Adhere®) polishing step (operated in flow through mode) resulted in over a five-fold increase in productivity with significant reduction in buffer usage compared to the use of a cation exchange chromatography (CEX) polishing step. The present invention further provides an alternative polishing step for resolving heterogenous aggregates comprising the use of hydrophobic interaction chromatography (e.g. Phenyl Sepharose HP) in flowthrough mode.

The HTS/DoE strategies investigated the effects of the experimental factors of buffer capacity, pH, conductivity, and column loading on the model responses of yield and monomer purity. Mathematical modeling was utilized to define a variety of design spaces and operating conditions, which led to the identification of a highly productive and cost effective hydrophobic-interaction-based chromatographic process for resolving a mixture of heterogeneous aggregates from a bulk drug preparation comprising an adalimumab biosimilar anti-TNF moloclonal antibody (referred to herein as “anti-TNF mAb B”). This process resulted in a 5-fold increase in productivity and 4-fold decrease in buffer cost per batch manufactured.

In certain embodiments, the present invention provides a method of purifying a monoclonal antibody in the presence of heterogeneous aggregates using mixed mode chromatography in flowthrough mode with the following feed and wash operating conditions: pH 6.8-7.7, conductivity of 4-25 mS/cm, protein loading of 100-205 grams of protein per liter of resin, and buffering capacity of 20-200 mM salt concentration. Preferred salts include but are not limited to sodium acetate, sodium phosphate, tris-HCl and sodium chloride. Optimized conditions that produced the best purity and yield of monomeric anti-TNF mAb B (e.g. adalimumab) comprised of the following parameters and ranges: pH 7.0-7.6, conductivity 10-20 mS/cm, protein loading of 125-205 grams of protein per liter of resin, and feed buffering capacity of 20-100 mM sodium acetate.

In certain embodiments, the method of the invention results in purification wherein the yield of monomeric monoclonal antibody is >80% and the monoclonal antibody is purified to a purity of ≧99.0% as assessed by high performance size exclusion chromatography (HP-SEC). In some embodiment, the method of the invention results in purification wherein the yield of monomeric monoclonal antibody is >85% and the monoclonal antibody is purified to a purity of ≧99.4% as assessed by high performance size exclusion chromatography (HP-SEC).

Using optimized conditions, the Capto Adhere® polishing step (operated in flow through mode) heterogenous aggregate content were reduced 6-fold from 2.5% to 0.4% at protein loading of up to 200 g/L CV, and the total yield of purified monomeric antibody was approximately 85%. At lower load of 100 g/L CV, the heterogeneous aggregate reduction was 25-fold from 2.5% to 0.1% and the total yield of monomeric was approximately 89%. The majority of the residuals were cleared in the Protein A and anion exchange chromatography steps. Implementation of the Capto® step in place of a cation exchange chromatography operation resulted in a 5× increase in chromatography productivity and allowed for a more continuous operation since the last two chromatography steps operated in flowthrough mode.

In one embodiment, the present invention provides a method of purifying a monoclonal antibody in the presence of heterogeneous aggregates using hydrophobic interaction chromatography (e.g. Phenyl Sepharose HP) in flowthrough mode with the following operating conditions: pH 7, conductivity of 110 mS/cm, protein loading of at least 425 grams of protein per liter of resin. Preferred salts include but are not limited to sodium phosphate, tris-HCl, and ammonium sulfate. This method of the invention results in purification wherein the yield of monomeric monoclonal antibody is ≧90% and the monoclonal antibody is purified to a purity of ≧99.5% as assessed by high performance size exclusion chromatography (HP-SEC). In preferred embodiments, using the methods of the invention, >90% of the host cell protein and DNA is removed and the yield of monoclonal antibody is at least >70%, more preferably >80%, >82%, >85%, >87%, or greater than >90%. In preferred embodiments, the monoclonal anti-TNF antibody is purified to a purity of at least about >90% to about >95% as assessed by high performance size exclusion chromatography (HP-SEC). More preferably, using optimized operating conditions the monomeric antibody is purified to a purity of >95% (i.e., 96%, 97%, 98%, 98.5%, 99%, 99.2%, 99.3%, 99.4%, 99.5%, 99.6%, 99.7%, 99.8%).

### BRIEF DESCRIPTION OF THE DRAWINGS

FIG. 1 provides a graphic display of percentage (%) of aggregates (open diamond) compared to concentration (starred line) of eluted anti-TNF mAb B (protein of interest) across an elution profile from a Poros HS50 cation exchange chromatography (CEX) column.

FIG. 2 Provides a graphic display of percentage of basic variants (solid triangle) compared to percentage of acidic variants (triangle marked with “+” symbol) as a function of column volume across a Poros HS50 cation exchange chromatography (CEX) column.

FIG. 3 Provides a graphic display of aggregate percentage (diamond marked with “+” symbol) as a function of the concentration of eluted adalimumab antibody (starred line) (protein of interest) across the elution profile from a Phenyl Sepharose HP hydrophobic interaction chromatography column. The secondary concentration peak contains the majority of aggregate species as detected by high performance-size exclusion (HP-SEC) chromatography.

FIG. 4 Provides a graphic display of percentage of basic variants (solid triangle) compared to percentage of acid variants (triangle marked with a “+” symbol), across the elution profile from a Phenyl Sepharose HP hydrophobic interaction chromatography column. The acidic charged aggregates elute first from the column since they are less hydrophobic than the basic charged aggregates.

FIG. 5 Yield on recovery (solid star) and HP-SEC purity (open diamond) results as a function of elution volume for bind-and-elute hydrophobic interaction chromatography. Pure anti-TNF mAb B was eluted for the first seven column volumes with a 95% yield indicating that the aggregates contain a higher hydrophobicity than the anti-TNF mAb B monomer and were easily separated from the monomer peak with minimal yield loss. This significant amount of 100% pure monomer in the majority of the elution peak indicated HIC can also be operated in a flowthrough mode to achieve higher productivities.

FIG. 6 Yield on recovery (solid star) and HP-SEC purity (open diamond) results as a function of product loading (g/L CV) for flowthrough hydrophobic interaction chromatography. Pure anti-TNF mAb B was eluted up to 200 g/L CV with an 85% yield. In addition, purity of anti-TNF mAb B was maintained at ≧99.6% up to at least 425 g/L CV loading indicating that HIC in flowthrough mode can effectively remove heterogeneous aggregates at significantly high-throughputs.

FIGS. 7A and 7B provides aggregation response surface plots as a function of protein loading and feed pH for the HTS of Capto Adhere® in initial design window at 3 mS/cm (FIG. 7A) and 15 mS/cm (FIG. 7B). Conditions to achieve a purity of ≧99.4% include pH≧7.0 at a protein loading of 60-120 grams of protein per liter of resin and conductivity range of 3-15 mS/cm.

FIGS. 8A and 8B provides yield response surface plots as a function of protein loading and feed pH for the HTS of Capto Adhere® in initial design window at 3 mS/cm (FIG. 8A) and 15 mS/cm (FIG. 8B). Conditions to achieve a yield of ≧70% include pH 5.6-7.2 at a protein loading of 100-140 grams of protein per liter of resin and conductivity range of 3-15 mS/cm.

FIGS. 9A-9C provide overlay plots of the specified response criteria limits as a function of feed pH and conductivity over different protein loading conditions of 100 g/L (FIG. 9A), 125 g/L (FIG. 9B), and 150 g/L (FIG. 9C). White color indicates the Capto Adhere® operation window based on the response criteria.

FIG. 10 Response surface plots of feed pH versus conductivity at a constant 125 g/L loading for model responses such as aggregate levels (a) and wash CVs (b). An increase in purity from 99.4% to 99.7% can result from an increase in feed pH from 7.2 to 7.7 and shows the optimal operating conditions are very robust in terms of achieving a purity of ≧99.4%. Wash column volumes increase significantly with conductivity and are minimized at a lower conductivity of 12 mS/cm.

FIG. 11 Graphic display of percentage of aggregates present in: the HIC flow through effluent (solid square); the CA effluent (solid triangle marked with “+”); the HIC effluent (solid inverted triangle); and the concentration of the POI in the CA effluent (solid diamond marked with “+”) versus the mass of POI loaded per liter of column volume (CV).

FIG. 12 Schematic representation of a chromatographic purification scheme used to resolve the heterogeneous aggregates present in a bulk drug comprising an adalimumab anti-TNF mAb biosimilar candidate.

### DETAILED DESCRIPTION OF THE INVENTION

As used herein, the term “adalimumab,” refers to a FDA approved fully humanized IgG1, TNF-alpha inhibitor monoclonal antibody (tradename Humira®) produced by Abbott Laboratories. Each IgG antibody molecule comprises two kappa light chains and two human IgG1 heavy chains, the total molecular weight of adalimumab is 148 kDa. Each light chain consists of 214 amino acid residues and each heavy chain consists of 451 amino acid residues. Adalimumab produced in CHO cells is characterized by a binding affinity of 100 mM to human TNFα, (U.S. Pat. No. 6,090,382).

As used herein, the term “reference product”, is used to refer to Adalimumab (HUMIRA). HUMIRA lot numbers 753459A41, 763489A40, 77367LJ08, 79385LJ40, 82420LJ40, 83439SP40, 85470LJ40, 864779A14, 86478LX40, 87008LX41, 87010XD03, 87497XD03, 87497XD16, and 91073LX40 were used herein for comparison purposes. Generally speaking, reference products are “innovator products” comprising an approved biopharmaceutical product which has been approved by a regulatory authority for marketing in a geographical region subject to its jurisdiction on the basis of a full regulatory submission establishing the efficacy, quality and safety of the originator product.

As used herein, the term “biosimilar” refers to a biopharmaceutical which is deemed to be comparable in quality, safety, and efficacy to reference product marketed by an innovator company.

As used herein, the terms “about” or “approximately” used with a pH or pi (isoelectric point) value refers to a variance of 0.1, 0.2, 0.3, 0.4 or 0.5 units. When used with a temperature value, “about” or “approximately” refers to a variance of 1, 2, 3, 4 or 5 degrees. When used with other values, such as length and weight, “about” or “approximately” refers to a variance of 1%, 2%, 3%, 4% or 5%.

As used herein, the term “aggregates” refers to protein aggregates. It encompasses multimers (such as dimers, tetramers or higher order aggregates) of the mAb to be purified and may result e.g. in high molecular weight aggregates.

As used herein, the term “misfolded mAb” refers to antibodies that are incorrectly or improperly folded thus altering the three-dimensional structure. Misfolds can also encompass the term “aggregate”. However, aggregates do not necessarily have to be misfolds.

As used herein, the term “acidic variant” refers to a variant of a target protein which is more acidic (e.g., as determined by cation exchange chromatography) than the target protein. An example of an acidic variant is a deamidated variant.

As used herein, the term “robust process” refers to a process that performs adequately within it operation parameters, consistently providing material of defined quality, purity and yield.

As used herein, the term “upstream process” refers to process steps associated with the production of a recombinant protein by culture and propagation of host cells. Upstream process considerations include clone selection methodologies, media selection, fed-batch culture operating conditions, culture feeding strategies.

As used herein, the term “downstream process” refers to process steps associated with the purification of a recombinant protein and removal of impurities.

As used herein, the term “cell culture supernatant” refers to a medium in which cells are cultured and into which proteins are secreted provided they contain appropriate cellular signals, so-called signal peptides. It is preferred that the recombinant mAb expressing cells are cultured under serum-free culture conditions. Thus, preferably, the cell culture supernatant is devoid of animal-serum derived components. Most preferably, the cell culture medium is a chemically defined medium.

As used herein, “clarified” refers to a sample (i.e. a cell suspension) having undergone a solid-liquid separation step involving one or more of centrifugation, microfiltration and depth filtration to remove host cells and/or cellular debris. A clarified fermentation broth may be a cell culture supernatant. Clarification is sometimes referred to as a primary or initial recovery step and typically occurs prior to any chromatography or a similar step.

As used herein, a “mixture” comprises an antibody of interest (for which purification is desired) and one or more contaminant, i.e., impurities. The mixture can be obtained directly from a host cell or organism producing the polypeptide. Without intending to be limiting, examples of mixtures that can be purified according to a method of the present invention include harvested cell culture fluid, cell culture supernatant and conditioned cell culture supernatant. A mixture that has been “partially purified” has already been subjected to a chromatography step, e.g., non-affinity chromatography, affinity chromatography, etc.

As used herein, the term “conditioned mixture” is a mixture, e.g., a cell culture supernatant that has been prepared for a chromatography step used in a method of the invention by subjecting the mixture to one or more of buffer exchange, dilution, salt addition, pH titration or filtration in order to set the pH and/or conductivity range and/or buffer matrix to achieve a desired chromatography performance. A “conditioned mixture” can be used to standardize loading conditions onto the first chromatography column. In general, a mixture can be obtained through separation means well known in the art, e.g., by physically separating dead and viable cells from other components in the broth at the end of a bioreactor run using filtration or centrifugation, or by concentration and/or diafiltration of the cell culture supernatant into specific ranges of pH, conductivity and buffer species concentration.

The terms “Chinese hamster ovary cell protein” and “CHOP” are used interchangeably to refer to a mixture of host cell proteins (“HCP”) derived from a Chinese hamster ovary (“CHO”) cell culture. The HCP or CHOP is generally present as an impurity in a cell culture medium or lysate {e.g., a harvested cell culture fluid (“HCCF”) comprising a protein of interest such as recombinant mAb expressed in a CHO cell). The amount of CHOP present in a mixture comprising a protein of interest provides a measure of the degree of purity for the protein of interest. Typically, the amount of CHOP in a protein mixture is expressed in parts per million relative to the amount of the protein of interest in the mixture. It is understood that where the host cell is another cell type, e.g., a eukaryotic cell other than CHO cells, an insect cell, or a plant cell, of a yeast cell, HCP refers to the proteins, other than target protein, found in a lysate of the host cell.

The terms “target protein” and “protein of interest” as used interchangeably herein, refer to a protein or polypeptide, including but not limited to, a recombinant monoclonal antibody that is to be purified by a method of the invention, from a mixture of proteins and, optionally, other materials such as cell debris, DNA, host cell proteins, media components, and the like.

The term “chromatography” refers to any kind of technique which separates an analyte of interest (e.g., a monomer mAb) from other molecules present in a mixture.

As used herein, the term “purified” is used to indicate that the relative concentration (weight of component or fraction divided by the weight of all components or fractions in the mixture) of a protein of interest (mAb) is increased by at least about 20%. In one series of embodiments, the relative concentration is increased by at least about 40%, about 50%, about 60%, about 75%, about 100%, about 150%, or about 200%. A component or fraction can also be said to be purified when the relative concentration of components from which it is purified (weight of component or fraction from which it is purified divided by the weight of all components or fractions in the mixture) is decreased by at least about 20%, about 40%, about 50%, about 60%, about 75%, about 85%, about 95%, about 98% or 100%. In still another series of embodiments, the component or fraction is purified to a relative concentration of at least about 50%, about 65%, about 75%, about 85%, about 90%, about 97%, about 98%, or about 99%.

The term “chromatography resin” or “chromatography media” are used interchangeably herein and refer to any kind of solid phase which separates an analyte of interest (e.g., an Fc region containing protein such as an immunoglobulin) from other molecules present in a mixture. Usually, the analyte of interest is separated from other molecules as a result of differences in rates at which the individual molecules of the mixture migrate through a stationary solid phase under the influence of a moving phase, or in bind and elute processes. Non-limiting examples include cation exchange resins, affinity resins, anion exchange resins, anion exchange membranes, hydrophobic interaction resins and ion exchange monoliths. The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

The terms “Protein A,” “ProA,” and “PrA” are used interchangeably herein and encompass Protein A recovered from a native source thereof, Protein A produced synthetically (e.g., by peptide synthesis or by recombinant techniques), and variants thereof which retain the ability to bind proteins which have a CH2/CH3 region, such as an Fc region. Protein A is generally immobilized on a solid phase support material. The term “ProA” also refers to an affinity chromatography resin or column containing chromatographic solid support matrix to which is covalently attached Protein A.

As used herein, the term “contaminant Protein A” is any type of functional, IgG binding offspring of a Protein A or a functional derivative thereof as defined above which is obtained upon eluting bound antibody from a Protein A affinity chromatography column. Such contaminant Protein A species may result e.g. from hydrolysis of peptide bonds which is very likely to occur by means of enzyme action in particular in industrial manufacturing. For example, dying cells in the cell culture broth or cells disrupted in initial centrifugation or filtration steps are likely to have set free proteases which can degrade the Protein A resin. This is particularly likely because Protein A chromatography is applied as an early step in downstream processing when the crudely purified, fresh product solution still harbors considerable protease activity.

As used herein, the terms “ion-exchange” and “ion-exchange chromatography” are used to refer to a chromatographic process in which a solute or analyte of interest (e.g., an Fc region containing target protein) in a mixture interacts with a charged compound linked (such as by covalent attachment) to a solid phase ion exchange material such that the solute or analyte of interest interacts non-specifically with the charged compound more or less than solute impurities or contaminants in the mixture. The contaminating solutes in the mixture elute from a column of the ion exchange material faster or slower than the solute of interest or are bound to or excluded from the resin relative to the solute of interest. “Ion-exchange chromatography” specifically includes cation exchange, anion exchange, and mixed mode ion exchange chromatography.

As used herein, the term “mixed-mode chromatography” refers to the use of solid phase chromatographic media that employ multiple chemical mechanisms to adsorb proteins or other solutes. Examples of mixed mode chromatographic supports include but are not limited to chromatographic supports that exploit combinations of two or more of the following mechanisms: anion exchange, cation exchange, hydrophobic interaction, hydrophilic interaction, hydrogen bonding, pi-pi bonding, and metal affinity.

As used herein, the term “capture step” refers to the first downstream processing step which captures the product of interest (POI) from the harvested culture media, concentrates the product, and achieves a first separation of the POI from impurities (e.g., cells, cell debris, DNA, host cell proteins).

The term “affinity separation,” or “affinity purification,” as used herein, refers to any purification or assaying technique which involves the contacting a sample containing a target analyte (e.g., an Fc region containing protein) with an affinity media (e.g., a solid support carrying on it an affinity ligand known to bind the analyte such as, for example, e.g., Protein A or a variant thereof) known to bind the target analyte.

The target protein generally retains its specific binding affinity for the biospecific ligand during the chromatographic steps, while other solutes and/or proteins in the mixture do not bind appreciably or specifically to the ligand.

As used herein, the term “polishing step” refers to a downstream processing step which occurs after the initial capture step and which is intended to remove smaller amounts of impurities that are present in the product stream and which are typically have more similarity to the product (i.e., monomeric antibody) than the impurities removed during the capture step (e.g., aggregated forms of the product, structural variants including misfolded product and modified product).

By “binding” a molecule to an chromatography resin is meant exposing the molecule to chromatography resin under appropriate conditions (pH/conductivity) such that the molecule is reversibly immobilized in or on the chromatography resin by virtue of ligand-protein interactions. Non-limiting examples include ionic interactions between the molecule and a charged group or charged groups of the ion exchange material and a biospecific interaction between Protein A and an immunoglobulin.

The term “specific binding” as used herein, such as to describe interactions between a target protein (e.g., an Fc region containing protein) and a ligand bound to a solid support (e.g., Protein A bound to a solid phase matrix or resin), refers to the generally reversible binding of a protein of interest to a ligand through the combined effects of spatial complementarity of protein and ligand structures at a binding site coupled with electrostatic forces, hydrogen bonding, hydrophobic forces, and/or van der Waals forces at the binding site. Generally, the greater the spatial complementarity and the stronger the other forces at the binding site, the greater will be the binding specificity of a protein for its respective ligand. Non-limiting examples of specific binding includes antibody-antigen binding, enzyme-substrate binding, enzyme-cofactor binding, metal ion chelation, DNA binding protein-DNA binding, regulatory protein-protein interactions, and the like.

As used herein, the term “non-specific binding” describes interactions between a molecule of interest (e.g., a target protein has described herein) and a ligand, or other compound bound to a solid support (e.g., Protein A bound to a solid phase matrix or resin), through electrostatic forces, hydrogen bonding, hydrophobic forces, and/or van der Waals forces at an interaction site, but lacking structural complementarity that enhances the effects of the nonstructural forces. Examples of non-specific interactions include, but are not limited to, electrostatic, hydrophobic, and van der Waals forces as well as hydrogen bonding.

The terms “flow-through process,” “flow-through mode,” and “flow-through chromatography,” as used interchangeably herein, to refer to a product separation technique in which at least one product (monomeric mAb) contained in a sample along with one or more contaminants is intended to flow through a chromatographic resin or media, while at least one potential contaminant or impurity binds to the chromatographic resin or media. The “flow-through mode” is generally an isocratic operation (i.e., a chromatography process during which the composition of the mobile phase is not changed).

As used herein, the term “buffer exchange step” refers to an in-line solution condition adjustment, which is typically an alternative in many conventional processes, to the use of a holding tank. In a typical buffer exchange step, two solutions can be mixed or titrated during transfer using solution blending in a pipe or mixing vessel, filtration device or apparatus. For example, a solution may be required to be diluted in order to reduce conductivity by blending the solution with another lower conductivity solution. Buffer exchange can be accomplished with the help of filtration devices, such as diafiltration, ultrafiltration and the like.

As used herein, the term “elute” refers to a process which removes a protein of interest from a chromatography resin by altering the solution conditions such that buffer competes with the molecule of interest for the ligand sites on the chromatography resin. A non-limiting example is to elute a molecule from an ion exchange resin by altering the ionic strength of the buffer surrounding the ion exchange material such that the buffer competes with the molecule for the charged sites on the ion exchange material.

The terms “bind and elute mode” and “bind and elute process,” as used interchangeably herein, refer to a product separation technique in which at least one product contained in a sample (e.g., an Fc region containing protein) binds to a chromatographic resin or media and is subsequently eluted.

The term “pooling strategy” and “pooling criteria” as used interchangeably herein, is used to describe the approach of combining and eliminating chromatography process eluate fractions to achieve target impurity clearance and enhance desired product quality attributes.

The terms “contaminant,” “impurity,” and “debris,” as used interchangeably herein, refer to any foreign or objectionable molecule, including a biological macromolecule such as a DNA, an RNA, one or more host cell proteins, endotoxins, lipids and one or more additives which may be present in a sample containing the mAb target protein that is being separated from one or more of the foreign or objectionable molecules using a process of the present invention. Additionally, such a contaminant may include any reagent which is used in a step which may occur prior to the purification process.

The “isoelectric point” or “pI” of a protein refers to the pH at which the protein has a net overall charge equal to zero, i.e. the pH at which the protein has an equal number of positive and negative charges. Determination of the pI for any given protein can be done according to well-established techniques, such as e.g. by isoelectric focusing.

As used herein, the term “buffer” refers to a solution that resists changes in pH by the action of its acid-base conjugate components. Various buffers which can be employed depending, for example, on the desired pH of the buffer are described in Buffers. A Guide for the Preparation and Use of Buffers in Biological Systems, Gueffroy, D., ed. Calbiochem Corporation (1975).

As used herein the term “loading buffer” refers to a buffer which is used to load the sample or composition comprising the target molecule of interest (e.g., an Fc region containing target protein) and one or more impurities onto a chromatography column (e.g., an affinity column or an ion exchange column). The loading buffer has a conductivity and/or pH such that the molecule of interest (and generally one or more impurities) is/are bound to the chromatography resin or such that the protein of interest flows through the column while the impurities bind to the resin.

An “intermediate buffer” is used to elute one or more impurities from the chromatography resin, prior to eluting the polypeptide molecule of interest. The conductivity and/or pH of the intermediate buffer is/are such that one or more impurity is eluted from the ion exchange resin, but not significant amounts of the polypeptide of interest.

The terms “wash buffer” and “equilibration buffer” are used interchangeably herein, refers to a buffer used to wash or re-equilibrate the chromatography resin prior to eluting the polypeptide molecule of interest. In some cases, the wash buffer and loading buffer may be the same.

An “elution buffer” is used to elute the target protein from the solid phase. The conductivity and/or pH of the elution buffer is/are usually such that the target protein is eluted from the chromatography resin. The term “isocratic elution” is used to refer elution condition in which the composition of the mobile phase is unchanged during the entire elution process.

Unless explicitly stated, reference to terms such as “a” or “an” is not limited to one. For example, “a cell” does not exclude “cells.” Occasionally phrases such as one or more are used to highlight the possible presence of a plurality. Reference to open-ended terms such as “comprises” allows for additional elements or steps. Occasionally phrases such as “one or more” are used with or without open-ended terms to highlight the possibility of additional elements or steps.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

Complete citations for references cited in this disclosure using an abbreviated format are provided in the reference list included with this application. The references cited in the present application are not admitted to be prior art to the claimed invention.

A systematic approach toward the creation of a “design space” understanding of a biopharmaceutical manufacturing process is essential for the design of a robust and well controlled manufacturing process. The combination of high throughput screening (HTS) and design of experiment (DoE) strategies were employed to rapidly evaluate different chromatographic modalities by investigating the effects of buffer capacity, pH, conductivity, and anti-TNF mab B column loading with model responses that included yield and monomer purity.

The application of HTS techniques coupled with design of experiment methodologies allows the exploration of a wide range of process variables and responses to quickly determine optimal operating zones using a best fit mathematical model.

A DoE model also can distinguish between main and interaction effects for each study variable and gives insight into which variables are statistically significant over the range studied given a specified response. Since DoE models also play an important role in late stage monoclonal antibody development for defining the operating and robustness spaces for a critical process variable, it is highly important to utilize these models in the beginning stages of development to not only define the optimal design space for each variable, but to also aid in process understanding for future development.

Mathematical modeling was utilized to define a variety of design spaces and operating conditions, which lead to the identification of a highly productive and cost effective chromatographic process for resolving a mixture of heterogeneous aggregates from a bulk drug solution comprising a biosimilar anti-TNF mAb to adalimumab (referred to herein as “anti-TNF mAb B) produced by CHO cell culture. Capto Adhere® resin operated in flowthrough mode was ultimately selected for use as a polishing step because of its hydrophobic capability, demonstrated ability to clearance process residuals (host cell protein, DNA, residual Protein A ligand) and potential for viral clearance (Gagnon 2009; Zhao, et al., 2009; Toueille, et al., 2011; GE Healthcare Application Notes; Fixler 2008; Eriksson et al., 2009).

Strategies for the development of a chromatography unit operation for the effective removal of antibody aggregates from a biopharmaceutical drug substance need to be evaluated on a case-by-case basis and require a consideration of the individual antibody as well each of the upstream manufacturing steps. The International Conference on Harmonization (ICH) Q8 Guidance document defines “design space” as “the multidimensional combination and interaction of input variables that have been demonstrated to provide an assurance of quality” (FDA, 2006, 2009, Jiang, C., et al., Biotechnology and Bioengineering 107(6):985 (2010). QbD involves three primary components: process knowledge, including a detailed understanding of process operational parameters/inputs and their impact on performance, the relationship between the process and a product's critical quality attributes and the association between a products quality attributes and its clinical properties (Biotechnology and Bioengineering 107(6):985 (2010).

The present invention utilized high-throughput screening (HTS) and design of experiment (DOE) methodologies to develop a simplified flow-through approach to separate heterogeneously charged aggregates present in an adalimumab conditioned mixture (bulk drug) obtained from CHO cell culture. The method described herein, uses a multi-modal chromatography resin in a flow-through-mode, and results in over a five-fold increase in productivity with significant reduction in buffer usage when compared to the traditional CEX method. An additional method described herein, uses a hydrophobic interaction chromatography resin in a flow-through mode, and results in over a ten-fold increase in productivity with significant reduction in buffer usage when compared to the traditional CEX method.

The methods of the invention can be used as a polishing step to purify adalimumab from any mixture containing the antibody. For example, antibody preparations to which the invention can be applied can include unpurified or partially purified antibodies from natural, synthetic, or recombinant sources. The mixture may be cell culture material, for example, solubilized cells, such as cell culture supernatant. It certain embodiments, it is a clarified cell culture harvest.

There are numerous condition/parameters of a monoclonal antibody (mAb) manufacturing process that can promote protein aggregation. For example, during a typical commercial manufacturing (e.g., production, purification, formulation, filling and storage operations) process an antibody is exposed to numerous environmental conditions and factors which can result in the formation of aggregates. Environmental conditions/bioprocess paramaters that can led to aggregation include, but are not limited to, pH, ionic strength, oxygen, temperature, protein concentration, shear forces from the use of pumps to maintain flow control during downstream processing steps, and exposure to external stresses, such as the interaction with metal surfaces, exposure to air, freezing and/or thawing may result in undesirable post-translational modifications or promote molecular unfolding which can promote aggregation. The presence, or absence, of certain ligands, for example, specific ions or counter ions may promote aggregation of a particular POI.

Therapeutic proteins are generally produced by cell culture using either mammalian or bacteria cell lines engineered to produce the POI by insertion of a recombinant plasmid containing the gene of that protein. Commerical scale cell cultures can produce antibody titers ranging from 1 to 5 grams/liter. The improved production levels in terms of antibody titer over the last 10 years has presented manufacturers with an increased likelihood of antibody aggregates. Published reports demonstrate that it is possible to influence the amount of aggregate produced during the cell culture and purification process by carefully controlling the environment (e.g., media components) and implementing appropriate strategies to minimize the likelihood of aggregation. In addition, purification steps such as anion exchange chromatography (AEX or CEX) or hydrophobic interaction chromatography (HIC) have been successfully used at a manufacturing scale to remove mAb aggregates from partially purified bulk drug.

During the production phase, protein aggregation can occur either within the cell following protein expression, or after the protein is secreted into the cell culture media (Cromwell et al 2006). Therefore, it is not surprising that protein aggregation during cell culture is a commonly reported issue, and aggregates levels as high as 30% have been reported for particular mAbs produced in mammalian cell culture (Biotechnol Bioeng 100(4):707, Kramarczyk, J F (2008).

The production phase is followed by a harvest step which is used to separate the host cells from the cell culture supernatant which comprises the secreted antibody. In a typical downstream processing scheme, a cell supernatant from cells used to produce a monoclonal antibody is clarified through the use of an initial purification step typically involving centrifugation or depth filtration. The clarified solution containing the monoclonal antibody is then separated from other proteins produced by the cell using a combination of different chromatography techniques, typically a capture chromatography step followed by a polishing chromatography step. Generally speaking, the chromatography steps separate mixtures of proteins on the basis of their affinity for a biological or biomimetic ligand, charge, degree of hydrophobicity, or size. A final filtration step typically involving ultrafiltration or sterile filtration yields the final product.

While various protein purification platforms are utilized in the pharmaceutical industry, a typical purification process for an antibody includes an affinity-purification step, such as Protein A affinity chromatography, followed by an anion exchange change (AEX) chromatography, then a final polishing step in bind and elute mode using cation exchange chromatography (CEX) or hydrophobic interaction chromatography (HIC).

The most widely used chromatography resin used to perform the capture step in a commercial antibody manufacturing process is Protein A which exploits a specific interaction between the antibody to be purified and an immobilized capture agent. Protein A is a 41 kD cell wall protein from Staphylococcus aureas which binds with a high affinity to the Fc region of antibodies. In practice, mAbs present in a cell-free harvest supernatant primarily and selectively bind to protein A via its Fc region and the impurities, such as host cell proteins (HCPs), DNA and endotoxins, remain unbound (chose et al., 2004; 2007). In practice, protein A affinity chromatography is not capable of resolving/removing aggregated antibodies from the desired antibody monomers present in the supernatant because multimeric forms and the monmeric form of the antibody are both likely to bind to the resin ligand. In addition, the use of protein A chromatography is associated with high cost. It is well-known that protein A capture steps should be performed using conditions which are selected to minimize the risk of the formation of antibody aggregates.

Antibody manufacturing processes typically include additional production scale chromatography steps in order to remove impurities present in the protein A eluate and to resolve protein aggregates. In particular, impurities such as host cell proteins (HCP), leached protein A, endotoxin, host cell DNA, and aggregates are removed during subsequent purification steps. In practice, commercial anion- and cation-chromatography resins, size exclusion chromatography, (SEC) resins, and/or hydrophobic interaction (HIC) resins can be used to both remove impurities and to separate antibody monomers from dimers and multimeric aggregates based on molecular charge, size, or hydrophobicity, respectively. Available purification processes may achieve the desired purity standards but often result in a trade off in cost or productivity.

Ion exchange chromatography has been demonstrated to be a useful production scale process which is capable of separating mAb monomers from dimers and larger multimeric aggregates present in a partially purified bulk product. It is well known that monoclonal antibodies have ionizable groups such as carboxyl groups and amino groups, and that the charge of these groups will depend on the pH. Therefore, depending on an antibody's isoelectric point (pI) the charge of a protein molecule can be manipulated by using exposing the bulk product to different pH conditions.

Ion exchange chromatography separates proteins by charge primarily through electrostatic interactions between charged amino acid side chains and the surface charge of the ion exchange resin. Protein retention has been explained as a “net charge” phenomenon in which a protein is considered to be a point charge and retention is a function of the net charge of the protein at the pH of the mobile phase (Kopaciewicz, 1983). The utility of ion exchange chromatography is attributed to the fact that when a working pH that is close to the POI's pI (around 0.2 logs lower than the target pI), the overall net charge of the POI (e.g., monomeric mAb) is low. In practice, the fact that aggregates will generally carry more charge than the product at the working pH range and therefore bind more strongly to ion exchangers than the monomeric form of the POI can be exploited as the basis of a production scale purification operation.

It has been reported that anion exchange chromatography, using an anion exchanger matrix such as Q Sepharose) can be used to resolve aggregates present in commercial antibody manufacturing processes (U.S. Pat. No. 6,177,548). Cation exchange (CEX) resin retains biomolecules by the interaction of sulfonic acid groups on the surface of the ion-exchange, and is generally operated in bind and elute mode at an acidic condition with sodium chloride as the primary elution component (Follman and Fahner, 2004). Molecules vary considerably in their charge properties and will exhibit different degrees of interaction with charged chromatography media according to differences in their overall charge, charge density and surface charge distribution. The charged groups within a molecule that contribute to the net surface charge possess different pKa values depending on their structure and chemical microenvironment. In most cases, the aggregates are relatively more basic when compared to the monomers, and therefore, are resolved at the tail end of the product peak. Typically, ion exchange chromatography steps are operated using parameters designed to ensure that the aggregates bind to the resin and the monomeric POI will flow-through.

Size exclusion chromatography (SEC) has also been reported to provide an effective process for reducing the level of aggregates present in a partially purified bulk product. The success of this type chromatography exploits the fact that the POI (monmeric mAb) has a smaller size than aggregates and elutes earlier. Published reports document the use of either Sephacryl 300 or Superdex 200 resins to achieve final product pools with less than 2% aggregates (Vazquez-Rey, M and Lang, D, Biotechnology and Bioengineering 108(7):1494 (2011). However, although it is technically feasible to reduce the level of aggregates present in a bulk drug solution using SEC, it is often not cost efficient to do so. Accordingly, the use of an SEC chromatography step is generally not considered to be a commercially feasible option for the removal of aggregates.

Another alternative chromatography operation that can be employed to separate monomers from aggregates is based on the fact that aggregation increases the hydrophobicity of the resulting molecular form relative to the hydrophobicity of the monomeric mAb. Monoclonal antibodies comprise of several different amino acids, and each amino acid has at least one amine and one acid functional group as the name implies. The different properties result from variations in the structures of different R groups. Side chains which have various functional groups such as acids, amides, alcohols, and amines will impart a more polar character to the amino acid. The degree of polarity of the amino acid depends on the ranking of polarity of the functional groups. For example, aspartic acid is more polar than serine because an acidic functional group is more polar than an alcohol group.

In practice, soluble high molecular weight (HMW) species almost universally exhibit stronger retention than the monomeric mAb protein of interest (POI). In practice, separation is accomplished the use of a gradients or increased volumes of buffer to selectively elute the POI from the resin. HIC is an established purification step for aggregate removal with the majority of aggregates eluting either on the tail portion of the native antibody peak or as a distinct peak (Lu, et al., 2009; Wang, et al., 2008; Queiroz, et al., 2001). Phenyl sepharose HP is usually the HIC resin of choice because of the particle size and known surface accessibility of aromatic residues on the monoclonal antibody structure, (Mahn, et al., 2005; Graumann and Ebenbichler 2005; Guide to Protein Purification by Murray P. Deutscher). In practice, chromatography operations based on hydrophobicity can be used for the removal of both process and product related impurities (Curr Pharma Biotechno 110(4):427 (2009).

Mixed mode chromatography media provide unique selectivities that cannot be reproduced by single mode chromatography methods such as ion exchange. Mixed mode chromatography provides potential cost savings, longer column lifetimes and operation flexibility compared to affinity based methods. However, the development of mixed mode chromatography protocols can place a heavy burden on process development since multi-parameter screening is required to achieve their full potential. Method development is complicated, unpredictable, and may require extensive resources to achieve adequate recovery due to the complexity of the chromatographic mechanism. Mixed mode chromatography refers to chromatography that substantially involves a combination of two or more chemical mechanisms. In some embodiments, the combination results in unique selectivities such that it is able to achieve fractionation among antibodies that cannot be achieved by a single mode support. In certain embodiments, the mixed-mode resin comprises a negatively charged part and a hydrophobic part. In one embodiment, the negatively charged part is an anionic carboxylate group or anionic sulfo group for cation exchange. Examples of such supports include, but are not limited to, Capto Adhere® (GE Healthcare).

Capto Adhere® is a strong anion exchanger with multimodal functionality which confers different selectivity to the resin compared to traditional anion exchangers. The Capto Adhere® ligand (N-Benzyl-N-methyl ethanolamine) exhibits multiple modes of protein-interactive chemistries, including ionic interaction, hydrogen bonding and hydrophobic interaction. The multimodal functionality of the resin confers it with an ability to remove antibody dimers and aggregates, leached protein A, host cell proteins (HCP), antibody/HCP complexes, process residuals and viruses. The resin is typically used in flow-through mode in the context of a production scale polishing step employing operational parameters designed to have the mAb pass directly through the column while the contaminants are adsorbed.

While alternative mixed-mode resins that do not comprise of a strong anion exchange group coupled with a hydrophobic aromatic group are not be expected to behave similarly to Capto Adhere®, optimal flow-through conditions for alternative mixed mode resin can be determined using the methods disclosed herein. Commercially available examples of alternative mixed mode resins include but are not limited to ceramic hydroxyapatite (CHT) or ceramic fluorapatite (CFT), MEP-Hypercel™, Capto-MMC™, Bakerbond™ Carboxy-Sulfon™ and Bakerbond™ ABx™ (J. T. Baker).

The chromatograph support may be practiced in a packed bed column, a fluidized/expanded bed column, and/or a batch operation where the mixed mode support is mixed with the antibody preparation for a certain time. A solid phase chromatography support can be a porous particle, nonporous particle, membrane, or monolith. The term “solid phase” is used to mean any non-aqueous matrix to which one or more ligands can adhere or alternatively, in the case of size exclusion chromatography, it can refer to the gel structure of a resin. The solid phase can be any matrix capable of adhering ligands in this manner, e.g., a purification column, a discontinuous phase of discrete particles, a membrane, filter, gel, etc. Examples of materials that can be used to form the solid phase include polysaccharides (such as agarose and cellulose) and other mechanically stable matrices such as silica (e.g. controlled pore glass), poly-(styrenedivinyl)benzene, polyacrylamide, ceramic particles and derivatives of any of these.

In some embodiments, the mixed mode support is packed in a column of at least 0.5 cm internal diameter and a height of at least 10 cm. Such embodiments are useful, e.g., for evaluating the effects of various conditions on a particular antibody. Another embodiment employs the mixed mode support, packed in a column of any dimension required to support preparative applications. Column diameter may range from less than 1 cm to more than 1 meter, and column height may range from less than 1 cm to more than 30 cm depending on the requirements of a particular application. Commercial scale applications will typically have a column diameter (ID) of 20 cm or more and a height of at least 25 cm. Appropriate column dimensions can be determined by the skilled artisan.

It is well known that one strategy which will allow increased amounts of antibody to be purified without an increased column volume is to increase the binding capacity of the chromatography step. Since buffer volumes are usually based on column volume, increasing the protein loading mass without increasing the column volume requires no increase in buffer volume. Considering residual impurities and product-related aggregates are often present at lower concentration, compared to the POI, significantly higher loading capacity is possible in flow-through mode. For HIC operation, use of a flow-through mode offer several advantages such as lower salt disposal cost and reduced tank corrosion. The reduced salt concentration also allows the use of increased POI concentration in the load without the risk of precipitation.

Flow-through mode applications are common in purification platforms to achieve high throughputs for antibody production. The most common flowthrough unit operation is anion exchange chromatography with extremely high throughputs achieved using membrane technology (Zhou et al., 2007; Glynn et al., 2010).

Protein heterogeneity, which increases the probability of multiple forms of a biopharmaceutical protein (e.g., a mAb) interacting with environmental factors that promote aggregation, is a contributing factor for the formation of aggregates during a commercial manufacturing process. It is well-known that there are several common causes for molecular heterogeneity in size. Molecular forms characterized by a smaller molecular weight than an intact antibody often result from enzymatic or nonenzymatic (chemical) cleavage, or incomplete formation of, or mispaired disulfide bridges. Larger molecular forms often result from molecular association, oligomer formation, aggregation or precipitation.

Heterogeneity in biopharmaceutical drug products can be introduced in every step of a commercial manufacturing process including, but not limited to the protein expression, recovery, purification, formulation and/or storage steps. The two main classes of heterogeneities are those that occur at the amino acid or peptide level (e.g., chemical modifications) and those that occur at the secondary and tertiary structural levels. Chemical and/or charge heterogeneity involves a modification of the primary sequence of the mAb. Common alterations which can occur during the manufacture of a biological drug substance include changes to the disulfide bonds, modifications in N-glycosylation, C-terminal lysine processing, glycosylation of Lys residues, deamidation, isomerization, oxidation, and hydrolysis/fragmentation In practice it is not uncommon for a first molecular alteration or modification to result in a second change/heterogeneity. For example, a charge variant caused by deamidation (which has the effect of introducing a negative charge) or oxidation, can lead to local protein conformational changes which makes it more likely that the modified mAbs will form complexes or aggregates.

IgGs are required to be N-glycosylated in the CH2 domain of the Fc to exhibit effector functions including antibody dependent cellular cytotoxicity (ADCC) and complement dependent cytotoxicity (CDC). This is because Fc glycosylation impacts antibody binding to Fc receptors and complement activating protein, C1q. Glycans found in the Fc are mainly complex biantennary structures with a high degree of heterogeneity containing different terminal sugars including sialic acid, galactose, N-acetylglucosamine and core fucose. Different terminal sugars may dramatically affect ADCC and CDC activities of antibodies. Modified glycosylation patterns which do not impact the pI of a biosimilar adalimumab antibody substantially (i.e., <0.2 units) are expected to have little or no effect on the mixed mode chromatography polishing step described herein.

Heterogeneity can also result from misfolding, posttranslational modifications or molecular degradation. Common post-translational modifications know to occur in IgG1 therapeutic monocolonal antibodies include deamidation, methionine oxidation, N-glycosylation, glycation, disulfide reduction or unusual linkage formation (Zhang, T et al., Journal of Chromatography A 1218:5079-5086 (2011). For example, there are several common modifications leading to the formation of charge variants on the antibody polypeptide chains, some of which occur enzymatically, such as N-terminal pyroglutamation (which adds a positive charge to a polypeptide by eliminating a negative charge of the side chain of glutamic acid), C-terminal lysine truncation, and proteolytic fragmentation. Nonenzymatic modifications can also contribute to the heterogeneity of an antibody drug substance, such as deamidation, partial structural unfolding, complex formation between the antibody and other host cell proteins or process impurities, and molecular aggregation. Because the presence or absence of C-terminal lysine residue(s) and N-terminal pyroglutamination occurs naturally, as evidenced by the presence of modified antibodies in human plasma, these modifications do not typically present a regulatory issue.

Aggregation is a general term that encompasses several types of interactions or characteristics. Aggregates of proteins may arise from several mechanisms and may be classified in numerous ways, including soluble/insoluble, covalent/noncovalent, reversible/irreversible, and native/denatured. Although protein aggregation is a common issue encountered during manufacture of biotherapeutics, the mechanisms of aggregation (i.e., oligomerization) are poorly understood. Protein aggregates can take on a variety of forms including fibrils, irregular or spherical particulates, skins, gels or a combination of one or more of these alternative forms. Aggregates can initially exist as small dimers or fragments which eventually develop into larger aggregates, such as sub-visible or visible particles. Depending upon the amino acid sequence of the biopharmaceutical and the conditions to which the protein is exposed during its production, purification, formulation and storage, protein molecules may be unfolded or partially unfolded.

Aggregates in therapeutic mAb drug products can be viewed as an evolving mixture of various types which can actively undergo transitional equilibrium states. At any given time, the heterogeneous molecular forms present in such a mixture may reach a new equilibrium and transform into an even larger aggregate or complex structure. Once the higher order structure of a protein is disrupted, non-native interactions of exposed hydrophobic regions can promote intermolecular interactions leading to aggregation, or in some cases, precipitation.

Protein aggregates can be classified in several ways, including soluble/insoluble, covalent/non-covalent, reversible/non-reversible, and native/denatured. Soluble aggregates refer to those that are not visible as discrete particles and that may not be removed by a 0.22 μm filter. Conversely, insoluble aggregates may be removed by filtration and are often visible to the unaided eye. Covalent aggregates arise from the formation of a chemical bond between two or more monomers, or result from the chemical linking of partially unfolded molecules with each other. Disulfide bond formation resulting from previously unpaired free thiols is a common mechanism for covalent aggregation. Oxidation of tyrosines may also result in covalent aggregation through the formation of bityrosine. Reversible protein aggregation typically results from relatively weak noncovalent protein interactions. The reversibility is sometimes indicative of the presence of equilibrium between the monomer and higher order forms. This equilibrium may shift as a result of a change in solution conditions such as a decrease in protein concentration or a change in pH.

There are numerous analytical methods available to the biopharmaceutical industry to detect, characterize, quantify and monitor aggregates in biopharmaceutical protein products. However, there is not a single established analytical method capable of evaluating all aggregates present in a given bulk drug solution. Generally speaking available test methods can be divided into two groups: methods that detect small aggregates such as dimers, soluble aggregates and protein fragments, and methods that detect large aggregates such as insoluble subvisible and visible particles. In practice it is typical to use combinations of analytical methods, either as orthogonal confirmatory methods (to compensate for the limitation of an individual method) or to perform a more powerful analysis. For example, use of a combination of mass spectrometry in combination with chromatography provides information on chemical and physical degradation.

Within the group of methods suitable for the detection of small aggregates, size exclusion chromatography (SEC) is routinely used to detect and monitor aggregates during lot release. Aggregation, or size heterogeneity, can alter not only the therapeutic, pharmacokinetic and pharmacodynamics profiles of biotherapeutic protein, but also has a negative impact on the safety profile, because it is considered a risk factor for immunogenicity. Aggregation of proteins may either reveal new epitopes or leads to the formation of multivalent epitopes, which may stimulate the immune system. Accordingly, the presence of aggregates of any type is typically considered to be undesirable because of the concern that the aggregates may lead to an immunogenic reaction (small aggregates) or may cause adverse events on administration (particulates) (Cromwell M E, et al. Protein Aggregation and Bioprocessing. AAPS Journal. 2006; 8(3): E572).

To ensure the safety of biopharmaceuticals, regulatory agencies, such as the Food and Drug Administration (FDA), impose stringent purity standards requiring that therapeutic proteins are substantially free from impurities, including product related contaminants, such as aggregates, incorrectly folded proteins, fragments, and variants of the recombinant protein, and process related contaminants, such as host cell proteins, media components, viruses, host DNA and endotoxins. However, a specific regulatory guidance document establishing acceptable levels of protein aggregates for biopharmaceutical proteins is currently not available. In practice, regulatory agencies expect manufacturers to be able to provide the agencies with data-supported justification for aggregate levels that are defined in their specifications.

To date, there are no established criteria describing how the Food and Drug Administration will require a biosimilar applicant to establish that a particular biopharmaceutical product is “highly similar” to a reference product. The statutory definition provides that a biosimilar product can have minor differences in clinically inactive components, provided that “there are no clinically meaningful differences between the biological product and the reference product in terms of the safety, purity, and potency of the product” (42 USC §262(i)(1)).

A robust biosimilar development program will include the accumulation of sufficient data regarding the quality, stability and molecular heterogeneity of a biosimilar product to predict the expected levels of molecular heterogeneity during its complete lifecycle. For some products, size heterogeneity may be a stability indicator for the particular drug product, for other biopharmaceutical products very small changes in the aggregate levels present in a drug product may adversely affect product efficacy and safety. A thorough understanding of the degradation pathways that are relevant to the stability of a particular biotherapeutic protein, process-related factors which may influence the heterogeneity in a bulk drug product and efficacy and safety data collected during preclinical and clinical development will enable a manufacturer to propose limits for product aggregate levels in its specification and to justify a control strategy. The nature of suitable control strategies to ensure the safety and quality of a biosimilar product will have to be determined on a case-by-case basis. In some instances, a control strategy could require the implementation of steps designed to minimize or inhibit aggregate formation during either upstream or downstream process steps. At a minimum a control strategy implemented to mitigate the risk of aggregates may require increased product monitoring for the characterization of molecular heterogeneity and the establishment of a minimal percentage of monomer specification required for lot release.

Adalimumab (HUMIRA®, Abbott Laboratories, Abbott Park, Ill., USA) is a fully human recombinant antibody which binds to human TNF-α. It was approved by the US Food and Drug Administration (FDA) in 2002 and by the European Agency for the Evaluation of Medical (EMEA) Products in 2003 for the treatment of rheumatoid arthritis. It was subsequently approved for the treatment of other TNF-mediated chronic inflammatory diseases, including psoriatic arthritis, chronic plaque psoriasis, ankylosing spondylitis, Crohn's disease and polyarticular juvenile idiopathic arthritis. It can be used alone or in combination with methotrexate (MTX) or other nonbiological disease modifying anti-rheumatic drugs (DMARDs).

Adalimumab was derived from murine monoclonal antibody MAK195 using guided selection phage display. The fully human, affinity matured clone D2E7, comprises human-derived heavy and light chain variable regions and a human IgG1 kappa (κ) constant region. Each IgG antibody molecule comprises two kappa light chains and two human IgG1 heavy chains, the total molecular weight of adalimumab is 148 kDa. Each light chain consists of 214 amino acid residues and each heavy chain consists of 451 amino acid residues. Adalimumab produced in CHO cells is characterized by a binding affinity of 100 mM to human TNFα, (U.S. Pat. No. 6,090,382).

For the treatment of rheumatic diseases, adalimumab is typically administered by subcutaneous injection at 40 mg every one or two weeks. It is marketed in prefilled syringes and as an autoinjection device called HUMIRA Pen), which is typically used by patients for self-administration. The prefilled syringes and autoinjector comprise 40 mg of adalimumab in 0.8 ml of a buffered solution of mannitol, citric acid monohydrate, sodium citrate, disodium phosphate dehydrate, sodium chloride and polysorbate 80.

The commercial manufacturing process for adalimumab comprises several chromatography steps, as well as a low pH treatment step and nanofiltration for virus inactivation/removal (EMEA European Public Assessment Report (EPAR), Scientific Discussion, published Mar. 30, 2006). Physico-chemical studies reveal that adalimumab is present in three major forms, corresponding to molecules carrying two, one or no C-terminal lysine. These three main molecular forms have been estimated to constitute about 85% of an adalimumab bulk preparation. The rest, representing approximately 15% of the bulk preparation, typically elutes as a number of poorly resolved peaks in an ion exchange HPLC assay. Despite extensive characterization of the more acidic species present in the three major forms of the adalimumab, no correlation between the shift and mobility and changes in antibody structure have been established by the innovator/originator. The EMEA concluded that because the species could not be resolved by traditional analytical methods (e.g. SDS PAGE), and their presence were demonstrated to not influence TNF-α binding in in vitro model systems it was likely that the structural differences are minor. The EMEA further concluded that the fermentation step is critical for the formation of the acidic species, and indicated that a combination of in-process fermentation controls and weak cation exchange (WCX) chromatography into regions for quantification constitutes an acceptable means of monitoring the presence of the more acidic molecular species for release specifications and for defining the stability of the product. (EMEA European Public Assessment Report (EPAR), Scientific Discussion, Published Mar. 30, 2006).

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### EXAMPLES

### Materials and Methods

Reagents:

Tris-HCl, sodium citrate, and tris base buffers were purchased from Hyclone (Logan, Utah). Sodium chloride, sodium phosphate, sodium acetate, ammonium sulfate and 50 wt % sodium hydroxide were obtained either from Fisher Scientific (Pittsburgh, Pa.) or Sigma Aldrich (St. Louis, Mo.). All other chemicals were analytical grade.

Antibody:

The monoclonal antibody (mAb) used for this study was a biosimilar antibody candidate for the humanized IgG1 antibody adalimumab (referred to herein as “anti-TNF mAb B”) produced in Chinese hamster ovary (CHO) cells. The cell culture supernatant was partially purified through Protein A chromatography and AEX and utilized as feedstock for the CEX, multi-mode (Capto Adhere®), and HIC experiments. The chromatography feedstocks contained representative levels of aggregates, host cell protein, residual DNA, and residual Protein A ligand.

Chromatography Columns and Resins:

The CEX column was packed with a strong CEX resin, Poros® 50HS particle size 50 μm (Applied Biosystems, Foster City, Calif.). Packed bed dimension was 1.1 cm internal diameter×10 cm length (column volume=9.5 mL). The HIC column was packed with strong HIC resin containing a phenyl ligand and a 34 μm particle size (GE Healthcare, Piscataway, N.J.). Packed bed dimension was 0.5 cm internal diameter 20 cm length (column volume=4 mL).

The mixed mode column was packed with Capto Adhere® resin containing a mixture of ligands, which included a quaternary amine, phenyl, and hydroxyl groups, and a 75 μm particle size (Applied GE Healthcare, Piscataway, N.J.). Packed bed dimension was 1.1 cm internal diameter 12 cm length (column volume=12 mL).

### Chromatography Operating Parameters:

A. Cation Exchange Chromatography

NaOH in the column storage buffer is washed out with 20 mM sodium acetate and 1M sodium chloride buffer for 3 CV at 110 cm/hr. The column is then equilibrated with 5 CV of 20 mM sodium acetate. Upon completion of the equilibration, the pH of the feed adjusted to the desired pH, and then loaded onto the column at the same flowrate. Following loading, the column is washed with the equilibration buffer for 5 CV at 110 cm/hr. The column loading is typically <25 g/L CV. The product is eluted via linear gradient, from 100% 20 mM sodium acetate to 100% 20 mM sodium acetate with 500 mM sodium chloride in 20 CVs. Collection of fractions was started at 100 mAU measured at 280 nm into the step. Following product collection the column is regenerated using 100% 20 mM sodium acetate with 1 M sodium chloride over 3 CV.

B. Hydrophobic Interaction Chromatography (HIC)

The column is equilibrated with 5 CVs of 1.3M ammonium sulfate, 50 mM sodium phosphate pH 7. The feed is adjusted to pH 7 using 1M tris base and conductivity is adjusted to either 142 mS/cm for bind and elute mode or 110 mS/cm for flowthrough mode using ammonium sulfate and then loaded onto the column at 300 cm/hr. In bind and elute operation, the column is washed with 5 CVs of the equilibration buffer and a 20 CV gradient from 100% to 0% equilibration buffer is performed to elute the monomer and aggregates from the column. Half volume column fractions are taken during elution to determine monomer purity and IEX profile. In flowthrough operation, the product collection begins at 50 mAU (absorbance measured at 280 nm) into the loading and ends at 350 mAU during the wash step. Product pool pH is 7 upon elution, and is quenched with 1M acetic acid to a final pH of 5.5 prior to analysis.

C. Multi-Modal Chromatography (Capto Adhere®)

The column is sanitized for 3 CVs using 0.1N NaOH and then equilibrated with 3 CV of 100 mM sodium citrate pH 3 followed by 5 CVs of 20 mM Tris-HCl, 110 mM NaCl pH 7.2. The pH and conductivity of the feed are adjusted to the desired values then loaded to the column at 300 cm/hr. Upon completion of the loading, the column is washed with 2-10 CV of equilibration buffer at the same flow rate. Because the product is in the flowthrough, the product collection begins at 100 mAU (absorbance measured at 280 nm) into the loading and ends at 350 mAU during the wash step. Product pool pH is 7.2 upon elution, and is quenched with 1M acetic acid to a final pH of 5.5 prior to analysis.

### High-Throughput Resin Screening:

Resin screening was conducted either at a microscale using a Tecan Genesis 150 (Tecan US, Research Triangle Park, North Carolina) coupled with a multi-variable design of experiment (DoE) plan or at a laboratory column scale using an AKTA Explorer 100 (GE Healthcare, Uppsala, Sweden) employing a bind elute or flowthrough operating mode. Column fractions were taken as necessary to determine monomer purity, aggregate charge heterogeneity, and overall yield.

Resin screening was conducted either at a microscale using a Tecan Genesis 150 (Tecan US, Research Triangle Park, North Carolina) coupled with a multi-variable design of experiment (DoE) plan or at a laboratory column scale using an AKTA Explorer 100 (GE Healthcare, Uppsala, Sweden) employing a bind elute or flowthrough operating mode. Column fractions were taken as necessary to determine monomer purity, aggregate charge heterogeneity, and overall yield.

The microscale high-throughput screening was performed using the slurry plate method of TECAN screening. The resin was pipetted by the TECAN into a 96-well filter plate containing a 1.2 um filter in a slurry percentage of about 30%. Fifty uL of resin slurry was dispensed into each well containing a miniature stir bar. The microplate was placed by the TECAN on a stirring station on the TECAN deck to for the subsequent screening steps where the resin was incubated for a period of ˜30 minutes for each step. Solutions were filtered from the resin using a vacuum pump and collected in 96-deepwell microplates for analysis.

Buffer and feed solutions were prepared by the TECAN using stock solutions containing up to 1M sodium chloride for conductivity adjustment at each pH in the screen in 100-mL troughs. Feed samples were pH adjusted by hand and placed on the TECAN deck in 15-mL Falcon centrifuge tubes. The solutions were pipetted into 96-deepwell plates on the TECAN deck for preparation and mixing using fixed tips. During the screen, the solutions were pipetted out of the plate at the equilibration, wash, and strip steps and dispensed into the slurry plate in a volume of 200 uL. The tips were flushed with water in between each aspirate/dispense cycle to minimize contamination. Two cycles were performed for the equilibration, load, wash, and strip steps. For the load steps, the filtrate was recovered in separate microplates. For all other steps, the filtrate from both cycles was collected in the same microplate.

Laboratory scale column screens were performed using column dimensions specified in the examples and an Akta Explorer. The columns were sanitized for 3 CVs using 0.1N NaOH and then equilibrated with the appropriate buffer prior to loading. All column steps were performed at 300 cm/hr at ambient temperature. Feed conductivity and/or pH adjustment was performed prior to the experiment and loaded onto the column to the desired loading. After loading, the columns were washed with 1-10 CVs of equilibration buffer. For columns operated in flowthrough mode, product collection begins at 50 mAU (280 nm) and ends between 50-350 mAU. For columns operated in bind and elute mode, elution comprised of a 20 CV gradient from 100% to 0% equilibration buffer.

### Analytical Methods:

### Protein Concentration Quantification:

Protein concentrations were determined using UV absorbance at 280 nm. The antibody extinction coefficient was 1.463.

### High Performance Size Exclusion Chromatography (HP-SEC):

A Tosoh TSK-GEL G3000 SWXL analytical size exclusion column (7.8 mm×30 cm, 5 μm, 250 Angstroms) was used quantification of antibody purity and aggregation percentage. The mobile phase was 250 mM sodium phosphate, 300 mM NaCl, pH 7; flow rate of 0.5 mL/min; column temperature of 25° C.

### High Performance Ion Exchange Chromatography (HP-IEX):

A Dionex ProPac WCX-10 column (4 mm i.d.×250 mm, 10 μm) was used for the 2D weak cation-exchange LC separation by applying a linear salt gradient of 20 mM sodium citrate pH 5.5/20 mM sodium phosphate, 95 mM sodium-chloride pH 8 with detection at 280 nm. Mobile Phase A: 20 mM sodium citrate pH 5.5; Mobile Phase B: 20 mM sodium phosphate, 95 mM sodium-chloride pH 8: From 55% to 100% B in 36 min; 6 min wash step at 100% B; and 14 min equilibration time at 45% B Flow Rate: 1 mL/min; Temperature: 35° C. Acidic variants eluted from the column prior to the main monomer peak while basic variants eluted after the main peak. Acidic or basic variant percentages were determined by the sum of the acidic or basic variant peak areas divided by the total peak area. The main monomer peak was determined by the main monomer peak area divided by the total peak area.

### Residual Impurity Assays:

Leached Protein A ligand levels were measured using an ELISA assay. Microtiter plates were coated with a chicken polyclonal antibody to ProA. After incubation, the plates were washed and a biotinylated chicken anti-ProA antibody was added. Binding of the ProA to the chicken antibody was detected with avidin-D conjugated to horseradish peroxidase (HRP). The peroxidase was detected by the addition of the enzyme substrate 3,3,5,50-tetramethylbenzidine (TMB). The solution absorbance was measured at 450 nm. The concentration of Protein ligand impurity in the test sample was interpolated from the standard curve using a four-parameter logistic function. Residual DNA levels in the product pools were tested using the Picogreen (Guilloa et al., J. of Chromatography A, (1113) Issue 1-2: 239-243, 2006) or QPCR assay methods. Host cell protein (HCP) levels in the product pools were determined using an in-house third generation Cygnus kit assay.

### Design of Experiment Software and Analysis:

Design Expert (Stat-Ease, Minneapolis, Minn.) was utilized to generate full factorial designs and model the response surface curvature to determine the optimal operating conditions for the multi-mode chromatography step. Model responses were fit to mathematical equations containing one or multiple variables as the main factors, plus their two factor interaction if statistically significant and none, one, or multiple variables as the quadratic term. The coefficient of determination (R2) values for these DoE models were ≧0.9 indicating the models contained a great fit to the actual data. The coefficient of variation (CV) percentage was relatively small for both the yield and purity models indicating the predicted values were closer to the actual values. The graphical prediction feature in Design Expert was used to define the operating conditions for the multi-modal chromatography step based on specified criteria for yield and monomer purity. These operating conditions were confirmed on both laboratory and pilot scales.

### Example 1

### Failure of CEX to Resolve Heterogenous Aggregates Present in Partially Purified Adalimumab Produced by CHO Cell Culture

### Purpose:

Evaluate the effectiveness of cation exchange chromatography as a final polishing step to resolve aggregates from anti-TNF mAb B. Determine the charge properties of the aggregates.

### Results & Discussion:

Typically, a monoclonal antibody (mAb) purification platforms contain a cation exchange chromatography (CEX) polishing step due to its ability to clear aggregate species based on charge distribution (Kelley, 2007; Lu et al., 2009; Maim et al., 2005). While being somewhat antibody dependent, purity numbers exceeding 99% are easily achievable with many different CEX resins and elution schemes (Shukla and Hinckley, 2008, Shukla et al., 2008, Shukla et al., 2001; Suda et al., 2009).

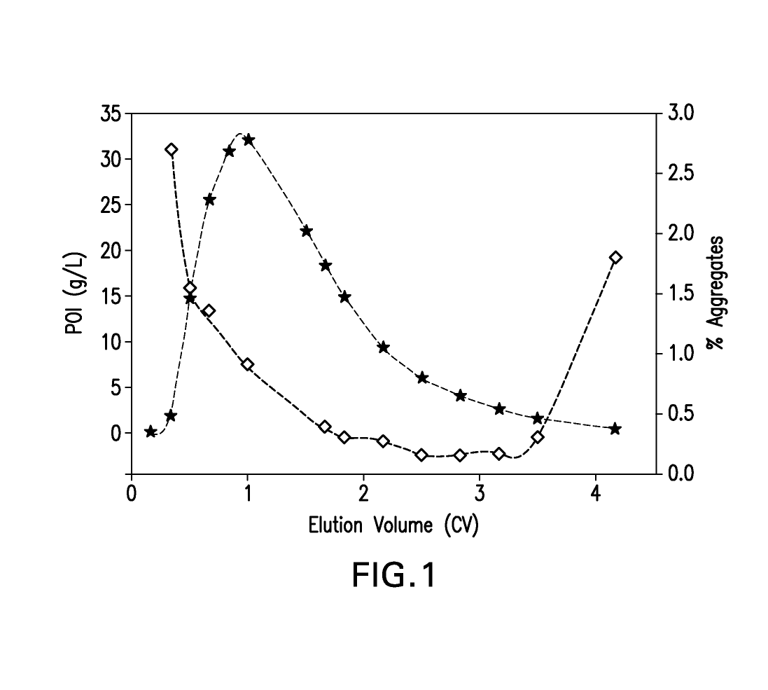

Accordingly, Poros 50 HS was the first resin evaluated for aggregate clearance of anti-TNF mAb B. Results of high-throughput-screening (HTS) indicated acceptable anti-TNF mAb B binding at pH 5.5. Anti-TNF mAb B was eluted using a linear sodium chloride gradient and was fractionated and analyzed in terms of aggregate content via HP-SEC. The percentage of aggregates present in the main peak comprising of the purified anti-TNF mAb B was observed to be characterized by an unacceptably high percentage of aggregates. The elution profile revealed about 2% of aggregates eluting in both the early and late portion of the anti-TNF mAb B peak (FIG. 1). Specifically, the analytical results revealed aggregates eluting at the tail end of the peak, as expected, but a significant portion of the aggregates were observed to co-elute with anti-TNF mAb B. This aggregate profile is significantly different from the more commonly reported distribution wherein aggregates are contained solely in the tail end of the elution peak.

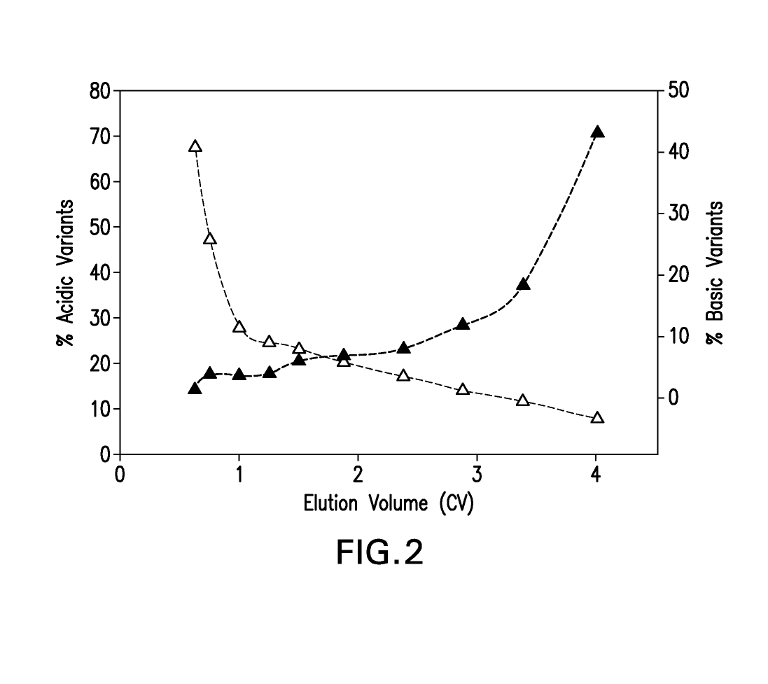

To understand this phenomenon, the peak fractions were analyzed via HP-IEX, and these analytical results indicated that about 60% of the aggregates were acidic variants, whereas 40% of the aggregates were basic variants (FIG. 2). This observation explains the aggregate elution profile, as CEX allows for a complimentary chemistry, charge-based elution, where mostly acidic, negatively charged species, elute first and basic, positively charged species, to elute towards the tail end of the anti-TNF mAb B peak. The early eluting aggregates are negatively charged acidic variants of the anti-TNF mAb B, and late eluting aggregates are positively charged basic variants. Since the clearance of these charge heterogeneous aggregates would involve shaving the elution peak at the front edge as well as the tail end, a bind and elute cation exchange chromatography step would drastically decrease the overall purification yield.

Aggregates of an acidic nature have not been widely reported in the literature. However, Zhou and co-workers made a similar observation during purification of their recombinant human monoclonal antibody using cation exchange chromatography and encountered similar selectivity challenges via the standard elution schemes. Zhou et al. (2007) describes the use of a pseudo-gradient elution method to resolve the aggregates via CEX. A major draw-back with the use of a pseudo-gradient to elute the product of interest is the requirement of having to shave both the front and back end of the product peak. This requirement compromises the robustness of the prior art CEX step and severely limits the utility of this approach.

### Conclusion:

Cation exchange chromatography operated in bind and elute mode showed the aggregates were composed of both acidic and basic charged variants. Acidic aggregates comprised 60% of the total aggregates and were observed to co-elute with the main product peak of monomeric anti-TNF mAb B, which limited the purification capacity of the CEX column. This variable distribution of acidic and basic aggregates in the anti-TNF mAb B CEX elution peak combined with the low protein loading capacity rendered the CEX polishing step unsuitable for a robust commercial purification strategy since HP-SEC purities of ≧99.4% could not be obtained with the CEX step.

### Example 2

### Characterization of the Hydrophobicity of the Anti-TNF mAb B Monomer and Aggregates and Evaluation of HIC Bind and Elute Polishing Step

### Purpose:

Evaluate the effectiveness of hydrophobic interaction chromatography in a bind-and-elute modality as a final polishing step to resolve heterogeneous aggregates from anti-TNF mAb B. Determine the hydrophobicity of the aggregates relative to the anti-TNF mAb B monomer.

### Results & Discussion:

Anti-TNF mAb monomers and aggregates were bound to a Phenyl Sepharose HP column at pH 7 and conductivity of 140 mS/cm and loaded onto the column up to 40 g/L CV. A linear elution gradient was employed with decreasing ammonium sulfate concentration from 100 to 0% over twenty column volumes (CVs) to separate the monomer from the aggregates by their hydrophobicity. The eluant was fractionated and tested for charge heterogeneity via HP-IEX and monomer purity via HP-SEC.

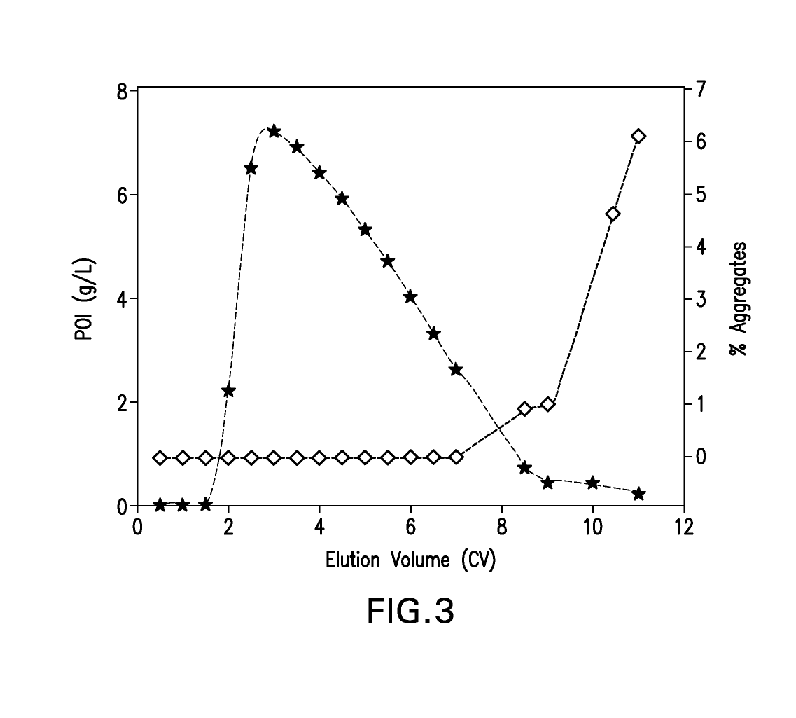

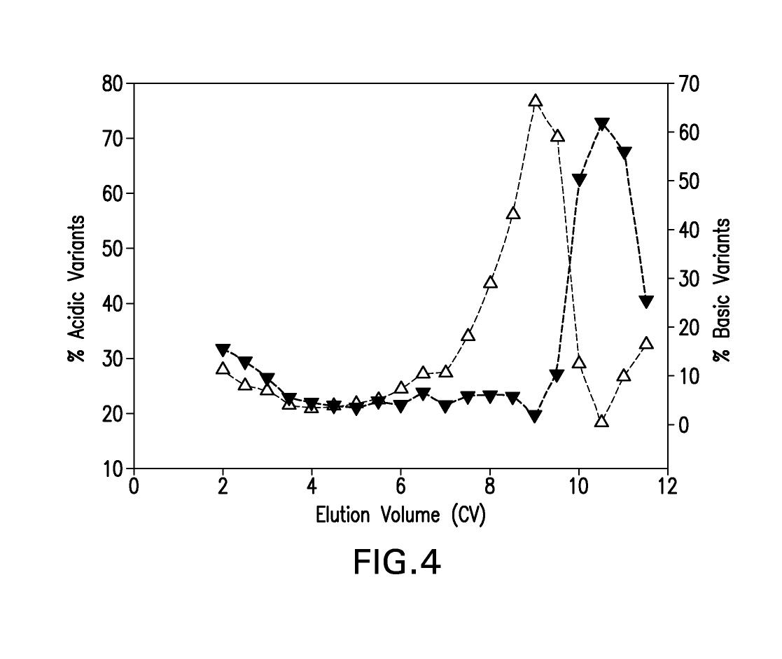

The chromatogram contained one main peak followed by a secondary smaller peak during the gradient elution as shown by the UV280 nm trace in FIG. 3. The main peak consisted entirely of high purity monomer while the secondary smaller peak contained increased levels of aggregate species, which included a mixture of both acidic and basic variants (FIGS. 3 and 4). This elution profile not only showed a clear separation of total aggregates from anti-TNF mAb B monomer but also separation of acidic and basic variant aggregate species due to their hydrophobicity relative to each other. The separation of anti-TNF mAb B monomer from aggregates due to their relative hydrophobicity correlates directly to the premise that monoclonal antibody hydrophobicity increases with the degree of aggregation since aggregate formation is known to take place through interactions between the relatively hydrophilic Fab regions of the IgG1 type mAbs (Wan et al., 2005). Studies have shown that the Fc region is more hydrophobic than the Fab regions (Nagaoka et al., 2001). Also, the Fc region of the mAb is neither involved in aggregate formation nor is affected by the process (Wan et al., 2005). This may explain why both the acidic and basic variants of the aggregates elute at a later time.

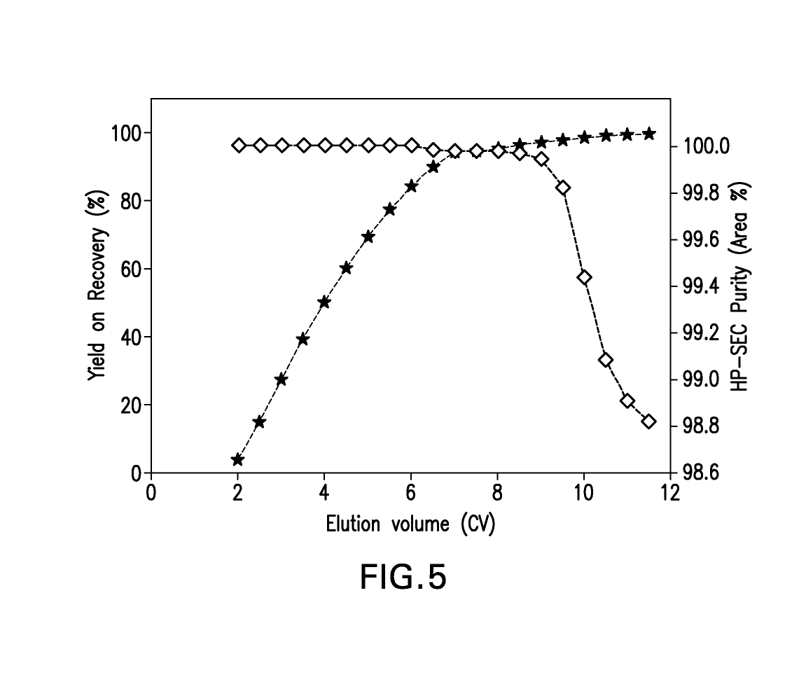

Since pure anti-TNF mAb B was eluted for the first seven column volumes with a 95% yield (FIG. 5), this indicated that HIC could be utilized in a bind-and-elute modality however the low step throughput coupled with ammonium sulfate disposal costs and unknown protein stability at high salt concentrations rendered the HIC polishing step unsuitable for a robust commercial purification strategy. On the other hand, this significant amount of 100% pure monomer in the majority of the elution peak indicated HIC can also be operated in a flowthrough mode to achieve higher productivities.

### Conclusion:

Bind-and-elute HIC modality exhibited superior separation of heterogeneously charged aggregate species from anti-TNF mAb B, which indicated these aggregates contained a higher hydrophobicity relative to the anti-TNF mAb B monomer. However, low binding capacity coupled with ammonium sulfate disposal costs and unknown protein stability at high salt concentrations rendered the HIC polishing step unsuitable for a robust commercial purification strategy. Since the majority of the elution peak contained 100% pure anti-TNF mAb B, then HIC operation in flowthrough mode was investigated as an alternative polishing step.

### Example 3

### Evaluation of a HIC Flowthrough Polishing Step

### Purpose:

Evaluate the effectiveness of hydrophobic interaction chromatography in a flowthrough modality as a final polishing step to resolve heterogeneous aggregates from anti-TNF mAb B.

### Results & Discussion:

Anti-TNF mAb monomers and aggregates loaded onto a Phenyl Sepharose HP column at pH 7 and conductivity of 110 mS/cm up to 425 g/L CV. After loading, the column was washed with an equivalent pH and conductivity buffer relative to the feed conditions to recover an additional unbound anti-TNF mAb B. Fractions were taken at various loading points to determine step yield and ant-TNF mAb B purity by HP-SEC.

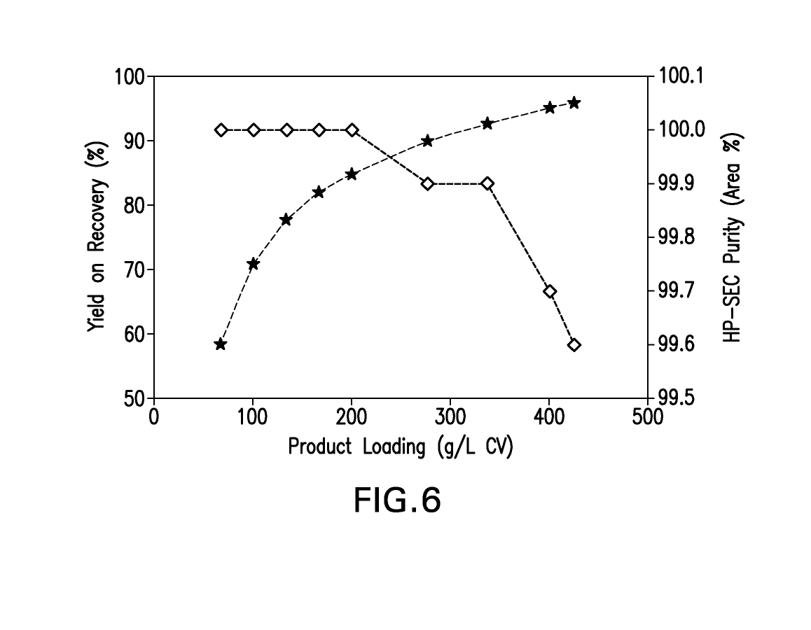

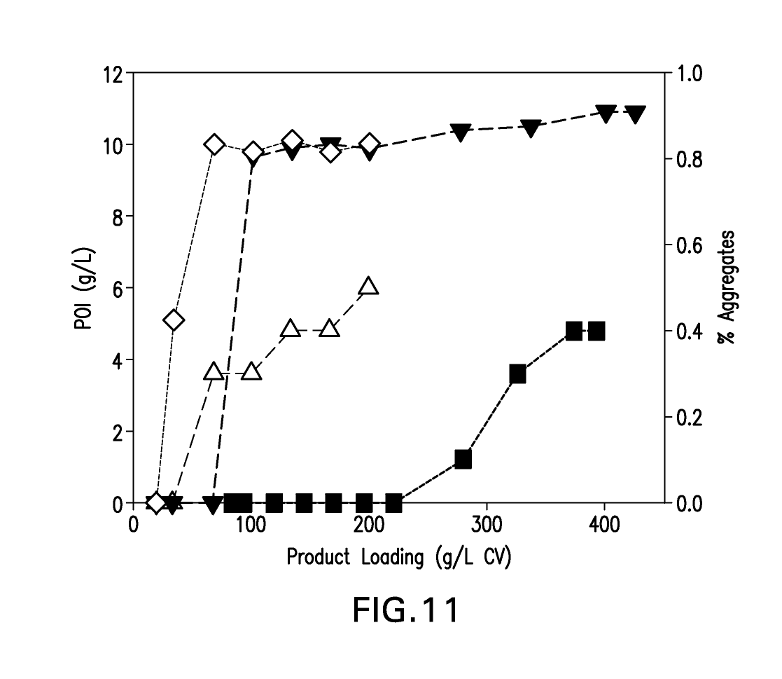

Product pools in flowthrough mode contained solely 100% pure monomer up to 200 g/L CV with an 85% yield (FIG. 6). In addition, purity of anti-TNF mAb B was maintained at ≧99.6% up to at least 425 g/L CV loading indicating that HIC in flowthrough mode can effectively remove heterogeneous aggregates at significantly high-throughputs (FIGS. 6 and 11). However, limitations of HIC still exist such as ammonium sulfate disposal costs and unknown protein stability at higher salt concentrations (even though the feed salt concentration required in flowthrough mode is 25% less than bind-and-elute mode). Since the main mechanism of aggregate clearance is hydrophobicity as shown by the HIC data, then the Capto Adhere® ligand containing hydrophobic properties was investigated for aggregate selectivity using a combination of high throughput and design of experiment methodologies. Advantages of Capto Adhere® flowthrough mode operation include using a lower salt concentration and different salt type in the feed composition for aggregate binding. Definition of the optimal Capto Adhere® chromatography conditions are described in the next example.

### Conclusions:

HIC operation in flowthrough mode resulted in over 10-fold increase in productivity and buffer usage while obtaining ≧99.6% purity of anti-TNF mAb B at >90% yield. However, limitations of HIC still exist such as ammonium sulfate disposal costs and unknown protein stability at higher salt concentrations. Since the main mechanism of aggregate clearance is hydrophobicity as shown by the HIC data, then the Capto Adhere® ligand containing hydrophobic properties was investigated for aggregate selectivity using a combination of high throughput and design of experiment methodologies. Advantages of Capto Adhere® flowthrough mode operation include using a lower salt concentration and different salt type in the feed composition for heterogeneous aggregate binding.

### Example 4

### Defining Process Design Space for a Capto Adhere® Flowthrough Polishing Step Utilizing a Combination of HTS and DoE Methodologies

### High-Throughput Screening:

### Purpose:

Capto Adhere® was operated in a bind-and-elute modality to determine the pH range required for the HTS design space. Based on the bind-and-elute results, the following design space was investigated using an automated liquid handling device (Tecan) coupled with DoE methodologies to rapidly determine initial Capto Adhere® operating conditions: pH 5.6-7.6, conductivity 3-15 mS/cm, and protein loading of 60-140 g/L CV.

### Results and Discussion:

Capto Adhere® resin was evaluated for aggregate selectivity based on the observation that a hydrophobicity-based method of separation could be used to resolve the heterogeneous aggregates present in a partially purified solution of anti-TNF mAb B. Capto Adhere® has advantages of using lower salt concentrations and different salt type versus HIC operation since it is operated in flowthrough mode. The Capto Adhere® resin design space was mapped using a combination of high throughput and design of experiment methodologies. The application of HTS and DoE allows for the exploration of a wide range of process variables and multitude responses to quickly determine optimal operating zones using a best fit mathematical model. A DoE model can also distinguish between main effects for each study variable and interaction effects. These methodologies were used for the Capto Adhere® design space due to the complex nature of the resin and potential interaction effects that may occur when using a multi-modal ligand. The evaluation included determining non-binding conditions, optimizing operating conditions such as feed pH, conductivity, and loading using the TECAN, verification of the TECAN conditions on a column, and optimization of operating conditions based on an expanded DoE and wash condition screens.

Capto Adhere® was initially evaluated in bind-and-elute mode in order to determine non-binding conditions and establish parameters for a TECAN screen. The bind-and-elute experiments were carried out using 1 mL HiTrap columns from GE Healthcare using an Akta Explorer 100 system. The feed material for the bind-and-elute experiments as well as the initial TECAN screen was Protein A product containing 0.8% aggregation determined by HPSEC. The feed was adjusted to pH 9.0 using 1 M trizmabase and loaded onto the column to approximately 5 g/L-resin. The buffer used for the bind-and-elute run was phosphate-citrate to allow for an elution gradient over a large pH range from 9.0 to 4.0. Elution of the product occurred with a peak maximum at pH 5.6. Therefore, pH 5.6 was used as the lowest pH in the TECAN screen with pH 7.6 being the highest recommended by GE Healthcare literature. The conductivity range was chosen from 3 mS/cm to 15 mS/cm based on the conductivity of the Protein A product for anti-TNF mAb B and previous mAb programs. The loading range was selected from 60-140 g/L based on parameters used for other mAb purification schemes.

The TECAN screen design space initially included four levels for feed pH (5.6, 6.6, 7.1, and 7.6), four levels for loading (60, 80, 100, and 140 g/L), and three levels for feed conductivity (3, 9, and 15 mS/cm) with monomer purity and yield as the main study responses. Buffers used for the screen were 20 mM phosphate for all pHs chosen except for pH 5.6 which used 20 mM citrate. A strip buffer of 100 mM citrate, pH 3.5 was used as a strip buffer in order to complete mass balances. The TECAN setup included duplication of each well to increase precision in the analysis.

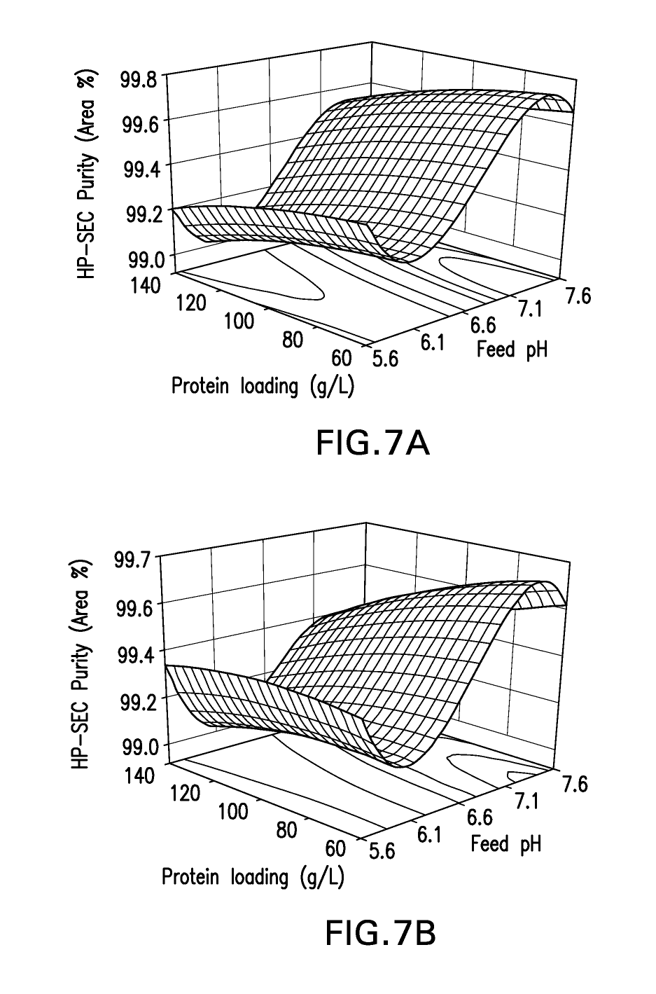

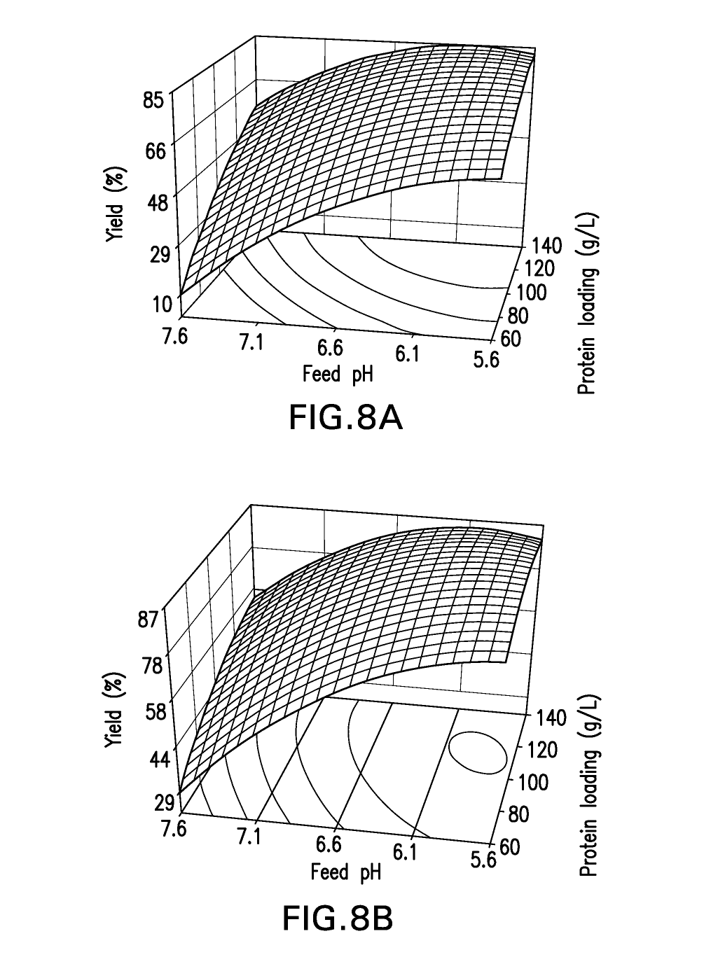

The product and aggregation levels in the filtrate plates are measured for each stream collected, two flowthrough plates and a strip plate, and used to generate the response surface maps in FIGS. 7-8. Design Expert software was used to predict the models for yield and aggregation clearance and to determine significant factors and interactions affecting the responses. The R2 values for these models were ≧0.9, which indicated the model contained sufficient accuracy. Factors returning p-values lower than 0.05 were considered significant in this method.

For aggregate removal, the model indicated pH and loading were significant factors. The plots in FIGS. 7A and 7B visualize these factors and indicate that lower loadings (<100 g/L_resin) and higher pH (6.8-7.6) operating conditions lead to the greatest aggregate reduction. Conductivity has a slight impact on aggregate clearance as well with the 3 mS/cm plot (FIG. 7A) showing an increased clearance compared to the 15 mS/cm plot (FIG. 8B).

The yield model (FIGS. 8A and 8B) showed that pH, conductivity, and loading were all significant factors affecting the yield response. Additionally, two factor interactions were identified as significant in the yield model in the pH-loading and conductivity-loading interactions. All of these factors and the factor interactions had p-values less than 0.01. The surface plots for the yield model show maximal yield at low pH and high loading conditions. Increasing conductivity allows for a greater operating window while still maintaining high product recovery of >80%. In order to balance the competing results of maximal yield and maintain the target reduction in aggregation, the centerpoint was chosen at pH 7.1, 9 mS/cm conductivity, and 100 g/L-resin loading.

Flowthrough parameters were tested using the 1 mL HiTrap column with feed containing a spike of induced aggregation material to challenge the column. Aggregation was induced by adjusting Protein A product to pH 1.8 using 1 N HCl and leaving the product in a 36° C. incubator over a ˜60 hour period, resulting in 80% aggregated material. This material was diafiltered into 20 mM acetate, pH 5.5 buffer and used as a spike into unchanged Protein A product in a ratio of 1:4. The resulting aggregation for the Capto Adhere® feed was ˜3%. The feed was adjusted to pH 7.1 using 1 M trizmabase and 5 M NaCl was spiked in to give a final conductivity of ˜9 mS/cm. Table 1 summarizes the flowthrough operating parameters and the results of the Capto Adhere® verification run.

TABLE 1
Capto adhere ® Operating Parameters and Verification Data

| Feed Conditions | | Product Results | |
|---|---|---|---|
| pH | 7.1 | | |
| Load (g/L-resin) | 100 | Product Collection | 100-100 mAu |
| Conductivity (mS/cm) | 9 | Yield (%) | 83 |
| Aggregate (%) | 3.05 | Aggregate (%) | 0.4 |

### Conclusions:

The surface plots in FIGS. 7A and 7B visualize these factors and indicate that lower loadings (<100 g/L_resin) and higher pH (6.8-7.6) operating conditions lead to the greatest aggregate reduction. The surface plots for the yield model (FIGS. 8A and 8B) show maximal yield at low pH and high loading conditions. Increasing conductivity allows for a greater operating window while still maintaining high product recovery of >80%. In order to balance the competing results of maximal yield and maintain the target reduction in aggregation, the centerpoint was chosen at pH 7.1, 9 mS/cm conductivity, and 100 g/L-resin loading. This condition was scaled to a 1 mL column and resulted in HP-SEC purity of 99.6% and 83% yield, which confirmed that the Tecan screening gave similar results for yield and purity compared to a column experiment.

### Expanded DoE Column Screen to Determine Robust Operating Conditions:

### Purpose:

HTS screening indicated the ability to produce a high purity (≧99.4%) anti-TNF mAb B monomer at various pH, conductivity, and protein loading conditions. An expanded DoE column screen was executed to define robust operating conditions for the Capto Adhere® step.

### Results and Discussion:

A two level full factorial study containing expanded conductivity (3.5-25 mS/cm) and protein loading (50-200 g/L) ranges was performed to assess robustness and determine Capto Adhere® operating conditions for scale-up. The experimental design also contained multiple levels of protein loading since this variable will contain the most variation upon scale-up. The performance parameters of the robustness study focused on chromatography yield and monomer purity of the Capto Adhere® product pool. The Protein A feed utilized for this study contained a 2.5% total aggregates. Target minimum values for yield and monomer purity responses were set at ≧85% yield and ≧99.4% purity.

Laboratory Capto Adhere® columns packed to a volume of 12 mL (1.1 mm×12 cm) were used to evaluate the expanded range study on an Akta Explorer. The columns were equilibrated to the specified pH and sodium chloride concentrations prior to loading Protein A product. After loading the Protein A product, the column was washed with equilibration buffer. The load eluate and wash volumes were collected separately as fractions and then combined as the product pool. The remaining bound proteins were eluted using a two phase regeneration procedure consisting of acidic and basic pH buffers.

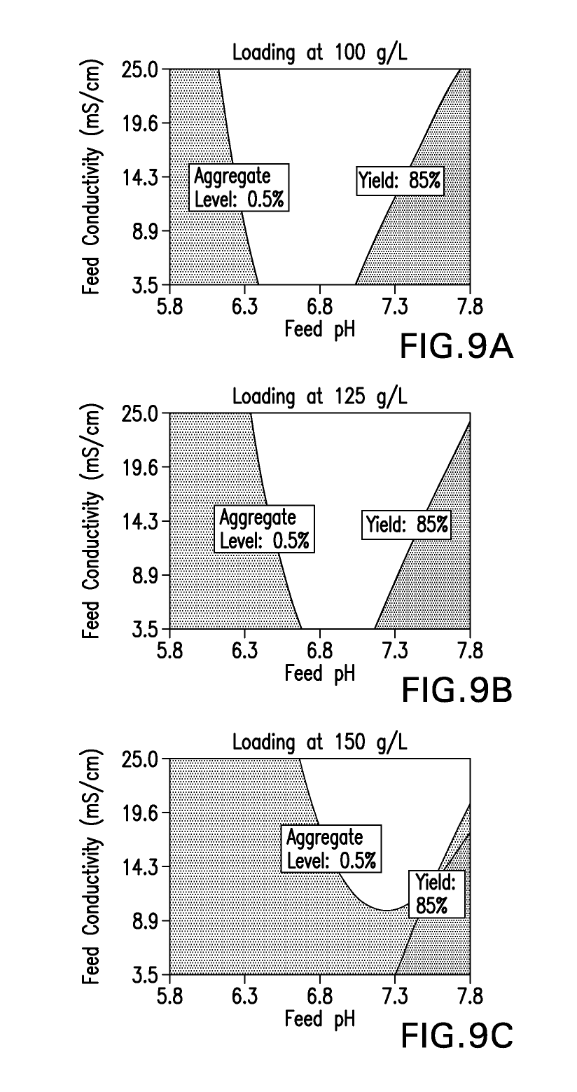

The yield and aggregate clearance trends were similar for the column DOE compared to the HTS results. The optimal operating window for the Capto Adhere® chromatography step was wider in the column screening compared to the HTS. As the protein loading increases from 100 to 150 g/L, the acceptable aggregate reduction zone shifts to basic pH values (FIG. 9). Salt is required in the feed for acceptable aggregate reduction at protein loadings of at least 150 g/L. The yield operating space remains fairly constant as the protein loading increases from 100 to 150 g/L. In order to balance maximizing Capto Adhere® productivity with maintaining step yield and purity targets, the following operating conditions were chosen and only slightly deviated from the initial HTS conditions: feed pH of 7.2, feed conductivity at 12 mS/cm, and protein loading of 125 g/L. This condition was verified on a laboratory scale column and resulted in 90% yield with 0.4% aggregates, which matched the predicted results from the mathematical modeling indicating high accuracy and precision of the model.

### Conclusions:

DoE methodology was then integrated with mathematical modeling to define the operating space in silico and center point process conditions for the multi-mode chromatography step. The yield and aggregate clearance trends were similar for the column DOE compared to the HTS results with the exception of a wider operating window discovered in the column screening compared to the HTS. The following multi-modal chromatography process conditions were defined: pH 7.2 (+/−0.2), 12 mS/cm (+/1), 125 g/L loading (+/−15 g/L). In addition to meeting the target for monomer purity at ≧99.4%, this condition resulted in at least 5-fold higher step productivity and at least 10-fold decrease in buffer usage compared to the CEX method and bind-and-elute HIC method. In addition, AEX can be operated in parallel with the Capto Adhere® and viral filtration steps in a continuous process mode if additional time cycle savings are desired.

### Buffer Capacity Screening & Increasing Capto Adhere® Throughput:

### Purpose:

Reduction of the wash column volumes (CVs) and aggregate clearance robustness were identified as step improvements. Both improvements were hypothesized to be a function of buffering capacity and wash/binding conditions (pH, conductivity). Two different buffer types (sodium phosphate versus Tris-HCl) were evaluated in the equilibration/wash buffer and one different buffer type (sodium acetate) was evaluated in the feed to determine the impact of buffering capacity on wash CVs. In addition, feed/wash pH and conductivity conditions were evaluated in a three level factorial design of experiment plan to determine wash CV robustness around the following levels: pH 7.2-7.7 and conductivity of 12-20 mS/cm with constant loading of 125 g/L and constant feed concentration of 100 mM sodium acetate.

### Results and Discussion:

The Capto Adhere® chromatography scale-up produced highly successful results in terms of step yield and impurity clearance (aggregates and residuals). Reduction of the wash column volumes (CVs) and aggregate clearance robustness were identified as step improvements. Both improvements were hypothesized to be a function of buffering capacity and wash/binding conditions (pH, conductivity). Two different buffer types (sodium phosphate versus Tris-HCl) were evaluated using a packed 4 mL Capto Adhere® column (0.5 mm×20 cm) on an Akta Explorer. The buffer type did not have a statistically significant impact on either yield, purity, or wash CVs (Table 2), so Tris-HCl was chosen due to its zwitterionic charge property.

TABLE 2
Buffer Type Comparison

| | Yield (%) | | Purity by RP-HPSEC (A %)* | |
|---|---|---|---|---|
| Wash Column | Sodium | | Sodium | |
| Volumes (CVs) | Phosphate | Tris-HCl | Phosphate | Tris-HCl |
| 0 | 73.1 | 72.6 | 99.6 | 99.7 |
| 1 | 79.5 | 78.4 | 99.3 | 99.4 |
| 2 | 85.3 | 81.7 | 99.5 | 99.5 |
| 3 | 86.3 | 84.6 | 99.8 | 99.6 |
| 4 | 87.4 | 86.2 | 99.8 | 99.6 |
| 5 | 88.4 | 87.7 | 99.8 | 99.7 |
| 6 | 89.3 | 88.8 | 99.8 | 99.6 |

*Purity results by RP-HPSEC are for the fractions only and not pool results. Yield results are pool results.

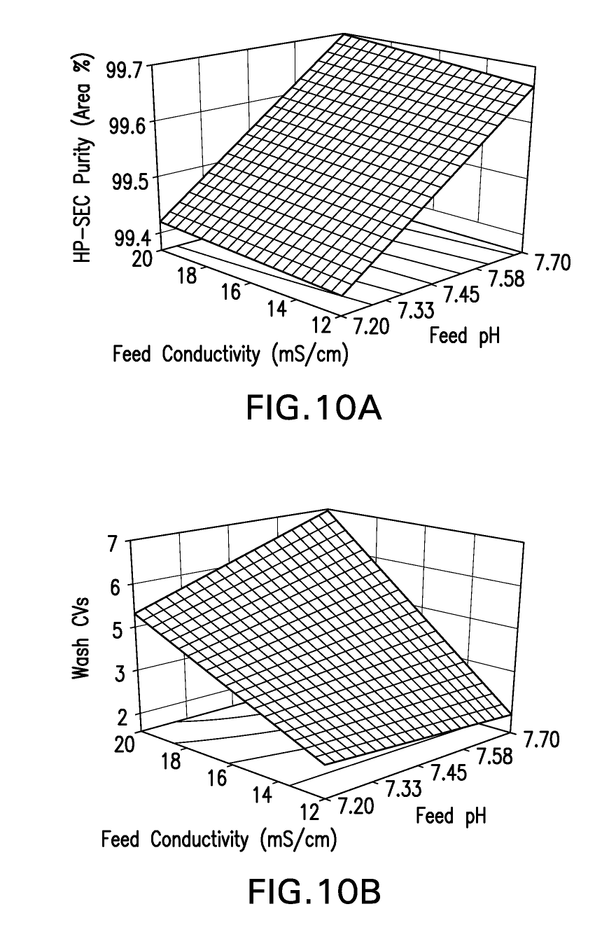

The feed buffering capacity was increased by addition of 100 mM sodium acetate and resulted in a significant reduction in wash CVs by 6-fold (from 12 to 2 CVs) while maintaining typical yields and purities of 88% and 99.4 A % respectively. This result indicated buffering capacity and perhaps buffer type were significant factors in wash CV reduction. However, previous screening showed the wash CVs can either increase with higher wash salt concentration at pH values of ≧7 or higher basic feed pH values. Both methodologies showed increasing wash CVs were shown to be independent of protein loading. Salt concentration in the feed enables higher step yields, (FIG. 10) especially at higher protein loadings. In addition, the model indicated an increase in feed pH may also lead to some additional aggregate reduction. Therefore, feed/wash pH and conductivity conditions were evaluated in a three level factorial design of experiment plan to determine wash CV robustness around the following levels: pH 7.2-7.7 and conductivity of 12-20 mS/cm with constant loading of 125 g/L and constant feed concentration of 100 mM sodium acetate. The study was performed using a 4 mL column (0.5 min×20 cm) and Akta Explorer system. Monomer purity by RP-HPSEC, step yield, and wash CVs were the main responses for this screen. Anion exchange product was utilized as the feed and contained representative levels of aggregate species.

Feed and wash salt concentrations had the most significant impact on wash CVs while pH displayed the most significant impact on aggregate clearance (FIGS. 9A-9C). Based on this modeling data, the feed center point conditions were updated to the following in order to provide an enhanced robustness for aggregate content and wash CVs: pH 7.4 (+/−0.2), 15 mS/cm (+/−1 mS/cm), 180 g/L loading (+/−25 g/L), 2 CV wash volume, 100 mM sodium acetate feed concentration. This condition ensures the wash CVs length will be maintained at 2 CVs in order to limit the dilution of the product pool. In addition, this updated operating condition provides a further robustness for aggregate content with levels ranging from 0.3 to 0.6% given a feed pH range of 7.2 to 7.6. Step yield only slightly decreased to 85% but increasing the protein loading to 180 g mAb/L resin CV resulted in a similar yield compared to scale-up performance while maintaining aggregate levels at approximately 0.5%.

### Conclusions:

Reduction of the wash column volumes (CVs) and aggregate clearance robustness were identified as step improvements. Feed pH, conductivity, and wash conditions were evaluated in a three level factorial design of experiment plan to determine wash CV robustness. Feed and wash salt concentrations had the most significant impact on wash CVs while pH displayed the most significant impact on aggregate clearance. Based on this modeling data, the feed centerpoint conditions were updated to the following in order to provide an enhanced robustness for aggregate content and wash CVs: pH 7.4 (+/−0.2), 15 mS/cm (+/−1 mS/cm), 180 g/L loading (+/−25 g/L), 2 CV wash volume, linear velocity range of 200-300 cm/hr. In addition, the Capto Adhere® feed contains 100 mM sodium acetate to prevent product tailing during the wash by maximizing feed buffering capacity. These MAEX conditions resulted in a 55 g/L loading increase, 6-fold decrease in wash CVs compared to previous conditions.

### Example 5

### Scale-Up Performance of Anti-TNF mAb Purification Using Capto Adhere® Polishing Step

### Purpose:

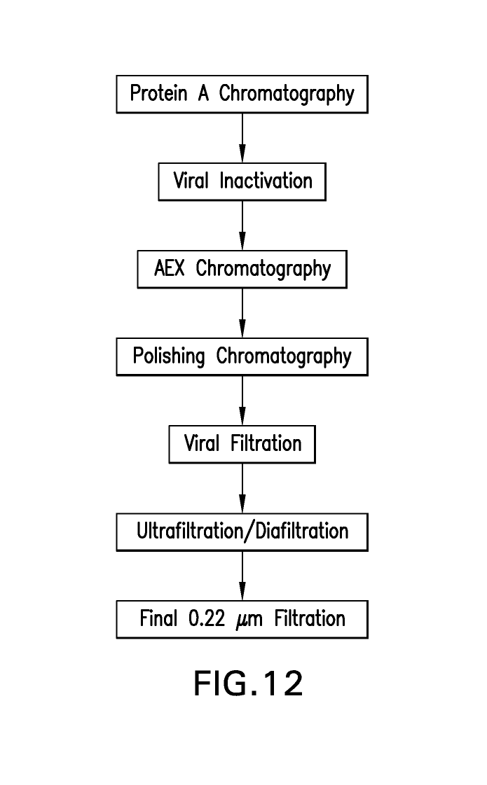

The anti-TNF mAb purification process (FIG. 12) was scaled at least 170-fold to assess the performance of the Capto Adhere® final polishing step upon scale-up.

### Results and Discussion:

For the first scale-up batch, a 7 cm inner diameter column was packed with Capto Adhere® resin to a height of 17.7 cm and final volume of 680 mls, which represented a 170-fold scale-up from previous laboratory experiments. The column was sanitized for 3 CVs using 0.1N NaOH and then equilibrated with 3 CV of 100 mM sodium citrate pH 3 followed by 5 CVs of 20 mM Tris-HCl, 110 mM NaCl pH 7.2. AEX product pH and conductivity were adjusted to 7.2 and 12 mS/cm respectively and loaded onto the column at a linear velocity of about 250 cm/hr for a total protein loading of 123 g/L CV. Feed properties are listed in Table 5. Upon completion of the loading, the column was washed with at least 2 CV of equilibration buffer at the same flow rate. Since the Capto Adhere® mode of operation is flowthrough, the product collection began at 50 mAU (absorbance measured at 280 nm) into the loading and ended at 400 mAU during the wash step. Product pool pH was 7.2 upon elution, and was quenched with 1M acetic acid to a final pH of 5.5 prior to analysis. The Capto Adhere® step performed similar to laboratory scale with a yield of 91%, HP-SEC purity of 99.5%, and residuals near below detection levels (Table 3).

The Capto Adhere® step was further scaled an additional 9-fold to a 6.1 L column (20 cm inner diameter, about 19.5 cm height) with two consecutive injections performed. The column was sanitized for 3 CVs using 0.1N NaOH and then equilibrated with 3 CV of 100 mM sodium citrate pH 3 followed by 5 CVs of 20 mM Tris-HCl, 110 mM NaCl pH 7.2. AEX product pH and conductivity were adjusted to 7.2 and 12 mS/cm respectively and loaded onto the column at a linear velocity of about 250 cm/hr for a total protein loading of 99.5 g/L CV and 110.8 g/L CV for each injection. Upon completion of the loading, the column was washed with at least 9 CV of equilibration buffer at the same flow rate. Since the Capto Adhere® mode of operation is flowthrough, the product collection began at 50 mAU (absorbance measured at 280 nm) into the loading and ended at 700 mAU during the wash step. Product pool pH was 7.2 upon elution, and was quenched with 1M acetic acid to a final pH of 5.5 prior to analysis. The Capto Adhere® step performed in similar fashion to the laboratory experiments with yields of 92% and 89% and HP-SEC purities of 99.9% and 99.6% per injection upon scale-up (Table 3).
The 680 mL Capto Adhere® column was utilized for the third and final scale-up batch and the anion exchange chromatography (AEX) and multi-mode chromatography were run in a continuous mode of operation. The column was sanitized for 3 CVs using 0.1N NaOH and then equilibrated with 3 CV of 100 mM sodium citrate pH 3 followed by 5 CVs of 20 mM Tris-HCl, 110 mM NaCl pH 7.4. AEX product was adjusted to 100 mM sodium acetate concentration by addition of 1M sodium acetate solution. AEX product pH was adjusted to 7.4 using 1M tris base and conductivity was adjusted to 15 mS/cm using 1M NaCl. This adjusted AEX product was loaded onto the column at a linear velocity of about 300 cm/hr for a total protein loading of about 180 g/L CV. Feed properties are listed in Table 3. Upon completion of the loading, the column was washed with 2 CV of equilibration buffer at the same flow rate to recover any unbound product. Product pool pH was 7.4 upon elution, and was quenched with 1M acetic acid to a final pH of 5.5 prior to analysis. The Capto Adhere® step performed similar to laboratory scale with a yield of about 85% and HP-SEC purity of 99.6% (Table 3).

TABLE 3
Capto adhere ® Scale-up Performance

| | | HP-SEC | | Protein A | DNA |
|---|---|---|---|---|---|
| Stream | Yield (%) | Purity (A %) | HCP (ppm) | Ligand (ppm) | (ppm) |
| Batch #1: Capto adhere | | 99.1 | 5.9 | 0.5 | 0.11 |
| feed | | | | | |
| Batch # 1: Capto adhere | 91 | 99.5 | 0.48 | Below | 0.09 |
| Product | | | | detection* | |
| Batch #2: Capto adhere | | 99.1, 99.2 | | | |
| feed | | | | | |
| Batch # 2: Capto adhere | 92, 89 | 99.9, 99.6 | | | |
| Product (Injections 1 & 2) | | | | | |
| Batch #3: Capto adhere | | 99.0 | | | 0.22 |
| feed | | | | | |
| Batch # 3: Capto adhere | 85 | 99.6 | | | Below |
| Product | | | | | detection** |

*Assay limit of detection is <0.1 ng/mL.
**Assay limit of detection is <1.9 ng/mL.

### Conclusions:

Capto Adhere® chromatography step performance at scale-up factors of 170-fold and over 1500-fold was similar to laboratory scale performance indicating a successful scale-up and a robust purification process.

### Example 6

### Differentiation of Multi-Modal Ligand Effects on Viral Clearance of Retrovirus and Parvovirus in the Anti-TNF mAb Purification Process

### Purpose:

Anion exchange chromatography (AEX) and multi-modal chromatography steps were tested for clearance of a retrovirus named Xenotropic murine leukemia virus (X-MuLV) and a parvovirus named murine minute virus (MMV). The Poros HQ50 anion exchange ligand contains a quaternized polyethyleneimine group while the Capto Adhere® multi-mode ligand contains both a quaternized polyethyleneimine group and phenyl group. In order to differentiate the viral clearance results of the two multi-mode ligands, an additional experiment was conducted by performing the AEX step with multi-mode chromatography feed conditions.

### Results and Discussion:

The ICH Q5A/Viral Safety Evaluation of Biotechnology Products Derived from Cell Lines of Human or Animal Origin guideline states that manufacturers of biologic products for human use should demonstrate the capability of the manufacturing process to remove and/or inactivate known viral contaminants. One method for assessing the viral safety of a biopharmaceutical is to perform a viral clearance study on the manufacturing process. This example will highlight the studies performed to determine the viral clearance capacity of the quaternized polyethyleneimine group compared to the phenyl group on the Capto Adhere® ligand for two viruses, X-MuLV retrovirus and MMV parvovirus.

The following viral clearance experiments were performed for the AEX and multi-mode chromatography steps for both X-MuLV and MMV. The virus spike for each experiment was 5 v % and the MMV contained an upgraded purity (Ultra 2). All experiments were performed at a linear velocity of 250 cm/hr.
1. AEX protein loading at both 100 g/L CV (duplicate runs per virus) and 200 g/L CV (single run per virus) at feed conditions of pH 7.2, 5 mS/cm
2. AEX performed using multi-mode chromatography feed conditions of pH 7.2, 12 mS/cm
3. Multi-mode chromatography protein loading at 150 g/L CV at feed conditions of pH 7.2, 12 mS/cm (duplicate runs per virus)

The viral clearance of either X-MuLV or MMV in the AEX step was not affected by the protein loading (Table 4). However, when the multi-mode chromatography feed conditions were performed on the AEX Poros HQ resin, the clearance of X-MuLV at >5.1 log₁₀ remained similar compared to the multi-mode chromatography X-MuLV clearance of 5.2 log₁₀ while the clearance of MMV decreased by approximately 4 log₁₀ compared to the multi-mode chromatography clearance (Table 4).

TABLE 4
Viral Clearance Performance for AEX & Multi-Mode Chromatography

| | Viral Clearance Results (log₁₀) | | | |
|---|---|---|---|---|
| Experiment | X-MuLV | | MMV | |
| AEX #1: 100 g/L protein loading | >6.67 | >6.31 | >6.33 | >6.82 |
| AEX #2: 200 g/L protein loading | >6.63 | | 7.43 | |
| AEX #3: Multi-mode | >5.11 | | 2.85 | |
| chromatography feed conditions | | | | |
| Multi-mode chromatography | 5.17 | >5.35 | >6.79 | >6.82 |

XMuLV is a larger, highly charged virus and binding will exhibit more salt tolerance than MMV which explains why the clearance of X-MuLV was similar for both the quaternized polyethyleneimine group and phenyl group on the Capto Adhere® ligand. MMV contains a weak surface charge and therefore a higher concentration of salt in the feed will inhibit binding of MMV to the quaternized polyethyleneimine group. This resulted in a MMV clearance of only 2.85 log₁₀. Furthermore, since the clearance of MMV was >6.8 log₁₀ for the multi-mode chromatography step, then the MMV binding was solely to the hydrophobic phenyl group on the Capto Adhere® ligand. Therefore, an additional 4 log₁₀ MMV clearance can be claimed for the multi-mode chromatography step. However, since X-MuLV clearance was consistent across both AEX #3 and multi-mode chromatography experiments then zero X-MuLV clearance can be claimed for the multi-mode chromatography step.

### Conclusion:

The viral clearance of either X-MuLV or MMV in the AEX step was not affected by the protein loading up to 200 g/L CV. XMuLV is a larger, highly charged virus and binding will exhibit more salt tolerance than MMV which explains why the clearance of X-MuLV was similar for both the quaternized polyethyleneimine group and phenyl group on the Capto Adhere® ligand. Therefore, zero X-MuLV clearance can be claimed for the multi-mode chromatography step. However, due to the weak surface charge of MMV, an additional 4 log₁₀ MMV clearance can be claimed for the multi-mode chromatography step.

Other embodiments are within the following claims. While several embodiments have been shown and described, various modifications may be made without departing from the spirit and scope of the present invention.

### REFERENCES

Cromwell M E, et al. 2006, Protein Aggregation and Bioprocessing. AAPS Journal. 8(3): E572).
Eriksson K, Ljunglof A, Rodrigo G, Brekkan E. 2009. MAb Contaminant Removal with a Multimodal Anion Exchanger: A Platform Step to Follow Protein A. Bioprocess International.
Fixler K. 2008. Viral Clearance Evaluation for Monoclonal Antibody Purification Processes Using Capto MMC and Capto Adhere Resins. 236th ACS National Meeting in Philadelphia.
Follman D, Fahrner R. 2004. Factorial screening of antibody purification processes using three chromatography steps without protein-A. J Chrom A 1024:79-85.
Gagnon P. 2009. IgG Aggregate Removal by Charged-Hydrophobic Mixed Mode Chromatography. Current Pharmaceutical Biotechnology 10: 434-439.
Ghose S. 2004. Preparative protein purification on underivatized silica. Biotechnol. Bioeng. 87(3):413-23.
Ghose S. 2007. Binding capacity differences for antibodies and Fc-fusion proteins on protein A chromatographic materials. Biotechnol. Bioeng. 96: 768-779.
Glynn J, Boyle D, Wealand J, Miller-Cary E, Chen B, Mensah P. 2010. Advances in Monoclonal Antibody Purification. BioPharm International Supplement.
Graumann K, Ebenbichler A. 2005. Development and Scale-up of Preparative HIC for the Purification of a Recombinant Therapeutic Protein. Chem. Eng. Tech. 28:
Jiang, C et al. 2010. Defining process design space for a hydrophobic interaction chromatography (HIC) purification step: application of quality by design (QbD) Principles.
Biotech. And Bioengineering 107(6):985
Kelley B. 2007. Very Large Scale Monoclonal Antibody Purification: The Case for Conventional Unit Operations. Biotechnol. Prog. 23: 995-1008.
Lu Y, Williamson B, Gillespie R. 2009. Recent Advancement in Application of Hydrophobic Interaction Chromatography for Aggregate Removal in Industrial Purification Process. Current Pharmaceutical Biotechnology 10: 427-433.
Mahn A, Zapata-Torres G, Asenjo J. A. 2005 A theory of protein-resin interaction in hydrophobic interaction chromatography. J. Chromatogr. A. 25:81-8.
Nagaoka S, Kanno M, Kawakami H, Kubota S. 2001. Evaluation of blood compatibility of fluorinated polyimide by immunolabeling assay, J. Artif. Organs 4: 107-112
Queiroz J A, Tomaz C T, Cabral J M S. 2001. Hydrophobic Interaction Chromatography of Proteins. Journal of Biotechnology 87: 143-159.
Shukla A, Hinckley P. 2008. Host cell protein clearance during protein-A chromatography: development of an improved column wash step. Biotechnol Prog 24:1115-1121.
Shukla A, Jiang C, Ma J, Rubacha M, Flansburg L, Lee S. 2008. Demonstration of robust host cell protein clearance in biopharmaceutical downstream processes. Biotechnol Prog 24:615-622.
Shukla A, Sorge L, Boldman J. 2001. Process characterization for metal affinity chromatography of an Fc fusion protein: a design-of-experiments approach. Biotechnol Appl Biochem 34:71-80.
Suda E, Thomas K, Pabst T, Mensah P, Ramasubramanyan N, Gustafson M, Hunter A. 2009. J. Chromatogr A 1216: 256-5264.
Suda E, Thomas K, Pabst T, Mensah P, Ramasubramanyan N, Gustafson M, Hunter A. 2009. Comparison of agarose and dextran-grafted agarose strong ion exchangers for the separation of protein aggregates. J. of Chromatogr A 1216: 5256-5264
Toueille M, Uzel A, Depoisier J F, Gantier R. 2011. Designing New Monoclonal Antibody Purification Processes Using Mixed-Mode Chromatography Sorbents 879: 836-843.
Vazquez-Rey, M and Lang, D. 2011 Aggregates in Monoclonal Antibody Manufacturing Processes. Biotechnol. And Biogengineering 108(7) 1494
Wan Y, Vasan S S, Ghosh R, Hale G, Cui Z F. 2005. Separation of monoclonal antibody alemtuzumab monomer and dimmers using ultrafiltration, Biotechnol. Bioeng. 90: 422-432
Wang W. 2005. Protein aggregation and its inhibition in bio-pharmaceutics. Int J Pharm 289 (1-2):1-30.
Wang L, Ghosh R. 2008. Fractionation of Monoclonal Antibody Aggregates Using Membrane Chromatography. Journal of Membrane Science 318: 311-316.
Zhao G, Dong X Y, Sun Y. 2009. Ligands for Mixed-Mode Chromatography: Principles, Characteristics, and Design. Journal of Biotechnology
Zhang, T et al. 2011 Isolation and characterization of therapeutic antibody charge variants using cation exchange displacement chromatography. Journal of Chromatography A1218:5079
Zhou J X, Dermawan S, Solamo F, Glynn G., Stenson R, Tressel T., Guhan S. 2007. pH-conductivity hybrid gradient cation-exchange chromatography for process-scale monoclonal antibody purification. J. Chromatogr A 1175: 69-80.

What is claimed is:

1. A method of purifying monomeric monoclonal antibody (mAb) from a conditioned mixture comprising one or more impurities and heterogenous aggregates comprising the steps of:
a) providing a sample comprising a mAb produced in a CHO cell expression system;
b) binding the mAb present in the sample to a Protein A affinity chromatography resin;
c) eluting the mAb from the Protein A resin, wherein the eluted product provides a second sample, optionally referred to as a Protein A pool (PAP);
d) binding the impurities present in the PAP to an anion exchange (AEX) chromatography resin;
e) collecting the flow through from step (d) wherein the flow-through provides a feedstock;
f) binding the impurities present in the feed solution to a polishing resin; and
g) collecting the flow through product from step (f) wherein the product provides a purified monomeric mAb composition.

2. The method according to claim 1, wherein the mAb is an anti-TNF biosimilar of adalimumab.

3. The method according to claim 2, wherein the polishing resin is selected from a mixed mode resin and a hydrophobic interaction chromatography (HIC) resin.

4. The method according to claim 3 wherein the polishing resin is Capto Adhere®.

5. The method according to claim 4, wherein the feedstock is loaded on the Capto Adhere® resin at about 100 grams to about 205 grams of protein per liter of resin, the pH of the feedstock is buffered at about pH 6.8 to about pH 7.7, the conductivity of the feedstock is about 4 m S/cm to about 25 mS/cm and the feed comprises a buffering capacity of about 20 mM to about 200 mM salt.

6. The method according to claim 5, wherein the pH of the feedstock is adjusted to pH 7.2 and the conductivity is adjusted to 12 mS/cm and the feedstock is loaded onto the resin at 125 grams of protein per liter of resin.

7. The method according to claim 7 wherein the yield of monomeric mAb is greater than 85% and the mAb is purified to a purity of greater than ≧99.0% as assessed by high performance size exclusion chromatography (HP-SEC).

8. The method according to claim 3 wherein the polishing resin is Phenyl Sepharose HP.

9. The method according to claim 7, wherein the feedstock is loaded on the Phenyl sepharose resin a about 200 grams of protein to about 425 grams of protein per liter of resin, the pH of the feedstock is buffered at about pH 7.0, and the conductivity of the feedstock is about 110 m S/cm.

10. The method according to claim 9, wherein the yield of monomeric monoclonal antibody is ≧90% and the monoclonal antibody is purified to a purity of ≧99.5% as assessed by high performance size exclusion chromatography (HP-SEC).

11. The method according to claim 10 wherein the yield of purified mAb recovered in step g) is greater than 90% and the mAb is greater than 99% monomer.

12. The method according to claim 3, wherein the impurities removed in step (d) comprises one or more negatively charged impurities selected from HCP, DNA and endotoxin.
